(19) United States
(12) Patent Application Publication
(10) Pub. No.: US 2024/0415450 A1
(43) Pub. Date: Dec. 19, 2024

(54) ALARMS AND ALERTS FOR MEDICATION DELIVERY DEVICES AND SYSTEMS

(71) Applicant: Bigfoot Biomedical, Inc., Milpitas, CA (US)

(72) Inventors: Bryan Mazlish, Palo Alto, CA (US); Sabine Kabel-Eckes, Mountain View, CA (US); Shannon Sieber, Santa Clara, CA (US); Jeff Boissier, San Jose, CA (US); George Crothall, Oceanside, CA (US); Yean Wah Chan, Irvine, CA (US)

(21) Appl. No.: 18/821,778

(22) Filed: Aug. 30, 2024

Related U.S. Application Data
(63) Continuation of application No. 17/444,003, filed on Jul. 29, 2021, now Pat. No. 12,076,160.
Application No. 17/444,003 is a continuation of application No. 16/335,163, filed on Mar. 20, 2019, now Pat. No. 11,096,624.
(60) Provisional application No. 62/433,124, filed on Dec. 12, 2016.

(51) Int. Cl. A61B 5/00; A61B 5/145

(52) U.S. Cl. CPC A61B 5/4839; A61B 5/002; A61B 5/14532; A61B 5/746; A61B 5/4848

(57) ABSTRACT

Systems, methods, and devices provide alarms and alerts in an on-body networked diabetes management system. Methods may include receiving glucose sensor data from a continuous glucose monitor and determining a dosage of insulin delivery based at least in part on the glucose sensor data. The method may include detecting an alarm or alert condition, and sending a wireless communication regarding the alarm or alert condition to a remote user-interface device. The method may include triggering an audible, visual, or haptic alarm or alert on the insulin delivery device unless an acknowledgement of the alarm or alert condition is received within a predetermined period of time.

### CROSS-REFERENCE TO RELATED APPLICATIONS

This application is a continuation of U.S. patent application Ser. No. 17/444,003 filed Jul. 29, 2021, which is a continuation of U.S. patent application Ser. No. 16/335,163 filed Mar. 20, 2019, now U.S. Pat. No. 11,096,624 issued Aug. 24, 2021, the disclosure of which is hereby incorporated herein in its entirety by this reference, which is a national phase entry under 35 U.S.C. § 371 of International Patent Application PCT/US2017/065894, filed Dec. 12, 2017, designating the United States of America and published in English as International Patent Publication WO2018/111927 A1 on Jun. 21, 2018, which claims the benefit under Article 8 of the Patent Cooperation Treaty to U.S. Provisional Patent Application Ser. No. 62/433,124, filed Dec. 12, 2016, the disclosure of each of which is hereby incorporated herein in its entirety by this reference.

### TECHNICAL FIELD

This disclosure relates to alarms and alerts for medication delivery devices and systems, particularly for medication delivery systems that have a medication delivery device that communicates with a primary user-interface for the medication delivery device and related systems and methods.

### BACKGROUND

People with Type I, Type II, or gestational diabetes must track their blood glucose levels and sometimes treat their condition to maintain appropriate blood glucose levels. Control of diabetes can include the monitoring of blood glucose levels using a blood glucose monitor (BGM) and sometimes a continuous glucose monitor (CGM). People with Type I, and some people with Type II or gestational diabetes, require insulin or an analog thereof. Because it cannot be taken orally, insulin is injected with a syringe or delivered subcutaneously by an external infusion pump. Excessive insulin delivery, however, can result in acute hypoglycemia, which can result in severe bodily injury and/or death. The failure to administer an appropriate amount of insulin to a person with diabetes, however, results in hyperglycemia, which can result in severe bodily injury and/or death. Because of the grave risks associated with diabetes, CGMs and insulin infusion pump systems typically provide a series of alarms and alerts that draw attention to the user's current glycemic condition, system conditions, and/or other potential issues, but these alarms and alerts can result in alert fatigue. Users having alert fatigue may start to ignore alarms or alerts or discontinue use of a CGM or insulin infusion pump, thus reducing the quality of their treatment. Moreover, users may wish to keep their external infusion pump or CGM concealed from view in order to avoid unwanted attention. Accordingly, there is a need for an improved system for providing diabetes-related information, alerts, and alarms.

### BRIEF SUMMARY

Medication delivery systems, methods, and devices provided herein include at least a medication delivery device (e.g., an insulin delivery device) and a remote user-interface device (e.g., a smartphone having an installed app) in communication (e.g., wireless communication) with each other. In some cases, the remote user-interface device can serve as the primary user interface for interacting with the medication delivery device, but the medication delivery device can be adapted to provide audible, visual, or haptic feedback under certain conditions. In some cases, methods, devices, and systems provided herein can have a controller in the medication delivery device detect an alarm or alert condition and send a wireless communication intended for a remote user-interface device prior to issuing an alarm or alert on the medication delivery device. In some cases, methods, devices, and systems provided herein can include a controller in the medication delivery device adapted to receive and a remote user-interface device adapted to send a wireless communication indicating that a user has acknowledged an alarm or alert condition. In some cases, methods, devices, and systems provided herein can include a controller in the medication delivery device adapted to cause the medication delivery device to provide an audible, visual, and/or haptic alarm or alert if the controller does not receive a user acknowledgement within a predetermined period of time after sending the wireless communication, which can ensure that the user is able to receive communications from the remote user-interface device. For example, in some cases, a remote user-interface device can be programmed by the user to not provide audible alarms, or the remote user-interface device may be too far away from the medication delivery device or may have a depleted battery. In some cases, the user acknowledgement can come to the controller via a wireless communication from the remote user-interface device or via user interaction with the medication delivery device.

Devices, methods, and systems provided herein can permit a user to stop or quiet an alarm or alert on the remote user-interface device and/or the medication delivery device by pressing one or more appropriate keys or user-selectable icons on the remote user-interface device (e.g., a snooze button) or the medication delivery device, or taking some other interactive action with the remote user-interface device and/or the medication delivery device (e.g., moving the remote user-interface device in a predetermined motion or series of motions). After an alarm or alert is stopped, in some cases, a controller in the medication delivery device may wait a predetermined snooze period of time before re-triggering an alarm or alert by sending a wireless communication intended for the remote user-interface device for the same alarm or alert condition. In some cases, a user will take appropriate action so that the alarm or alert condition is resolved or resolving such that the alarm or alert condition will not be present after the snooze period. In some cases, different alarm or alert conditions will have different predetermined periods of time based on a danger associated with the alarm or alert condition. In some cases, each alarm or alert condition will have the same predetermined periods of time regardless of the danger associated with the alarm or alert condition. For example, in some cases, an alarm for severe hypoglycemia may only be snoozed for a limited amount of time (e.g., between 5 and 10 minutes), while an alarm for hypoglycemia may be snoozed for a relatively longer period of time (e.g., between 15 and 30 minutes). In some cases, a user can snooze and/or acknowledge an alarm or alert condition via a button on the housing of the medication delivery device. In some cases, medication delivery devices provided herein can include a tap detector and/or a proximity sensor and a user can snooze and/or acknowledge an alarm or alert condition by tapping on the housing of the medication delivery device and/or by motioning ((e.g., with the user's hand) within a selected proximity of the delivery device). In some cases, medication delivery devices provided herein will only recognize an acknowledgement via the medication delivery device if there is a predetermined number or pattern of taps, button pushes, or gestures, which can prevent inadvertent button presses or jostling of the medication delivery device from silencing an important alarm. In some cases, alarms or alerts on either the medication delivery device, the remote user-interface device, and/or another remote device can be acknowledged/silenced from one or more of the devices. For example, in some cases a user may hear an audible tone from their remote user-interface device and wish to silence it via quick button press or tap on the medication delivery device even before any audible, visual, or haptic alarm or alert is issued on the medication delivery device. In such cases, the controller in the medication delivery device can send a wireless communication of the acknowledgement to the remote user-interface device to stop the alarm tone. In some cases, the snooze period can depend on the type of alarm or alert. In cases, the snooze period can be the same length of time for all alarm or alert conditions. In some cases, the snooze period can be for a selected period of time, for example, between about 5 minutes and about 30 minutes, between about 10 minutes and about 20 minutes, or about 15 minutes.

A remote user-interface device of systems and methods provided herein can be an intended primary user interface, to provide users with the convenience and discretion offered by the use of remote user-interface devices, such as the user's smartphone. In some cases, methods and systems provided herein can limit the ability of a user to control the medication delivery device without the presence of the remote user-interface device. For example, in some cases, a user will not be able to instruct the medication delivery device to deliver additional medication (e.g., a bolus of medication) without the remote user-interface device. In some cases, the primary user interface can be an application downloadable onto a user's smartphone and adapted to be paired to the medication delivery devices provided herein. Restricting the ability of a user to control the medication delivery device directly may seem counter-intuitive, but redirecting the user's attention to a remote user-interface device can conserve the power supply in the medication delivery device by reducing the power needed to power a robust user interface, as the medication delivery device may be a medication infusion device, intended to be worn on the user's body to provide regular, near continuous, or continuous delivery of medication to the user based on the user's needs, which can make the recharging of a battery in the medication delivery device inconvenient to a user. Remote user-interface devices, such as a smartphone, however, can be more convenient to recharge, and thus can be used to provide better graphics, better sound, and a more intuitive user experience. Additionally, a more robust user experience provided on a remote user-interface device can reduce the opportunity for a user to make a mistake in the delivery of medication. Also, a remote user-interface device can permit users to check the status of a medication delivery system provided herein without needing to directly access their medication delivery devices.

In some cases, methods, devices, and systems provided herein can additionally include an analyte sensor. For example, in some cases, an insulin delivery system can include a continuous glucose monitor. In some cases, an analyte sensor can be in wireless communication with the medication delivery device and/or the remote user-interface device. In other cases, an analyte sensor can be part of a medication delivery device. In some cases, medication delivery methods, devices, and systems provided herein can automate the delivery of medication to the user based on data from one or more analyte sensors. Automating medication delivery based on feedback from an analyte sensor, however, is only possible if the analyte sensor is providing actionable data. Accordingly, in some cases, methods, devices, and systems provided herein can include a medication delivery device having multiple modes of operation including at least one automation mode (e.g., where medication dosages or rates are changed depending at least in part on data from the analyte sensor) and at least one non-automation mode (e.g., where medication is delivered according to a programmed rate or dosage schedule). Accordingly, medication delivery methods, devices, and systems provided herein can include a medication delivery device adapted to inform the user regarding a current mode of operation even without the presence of the remote user-interface device. For example, in the case of a person with diabetes (PWD) using an insulin delivery system having an automated mode that changes basal rates based on data from a continuous glucose monitor (CGM) and a mode that simply delivers according to a schedule, the PWD may want to be more cognizant of their blood glucose levels if that PWD knows that the system is not actively adjusting basal rates based on CGM data. Additionally, methods, devices, and systems provided herein can be configured to indicate whether actionable data from the CGM is being received by the medication delivery device on the medication delivery device. In some cases, a housing of the medication delivery device can include a light that indicates the current mode of the medication delivery device. In some cases, the light can be adjacent to a mode icon or can illuminate a mode icon.

In some cases, methods, devices, and systems provided herein can alert the user to a need to take additional action to correct for an analyte condition based on data from an analyte sensor. For example, in some cases, an insulin delivery system provided herein in wireless communication with a continuous glucose monitor can provide a PWD with a notice regarding a need to correct a current or anticipated hypoglycemic condition by consuming carbohydrates or a notice regarding a need to administer additional insulin to correct a current or anticipated hyperglycemic condition. In some cases, methods, devices, and systems provided herein may include a user interface on the medication delivery device that does not provide a specific or relative analyte concentration, but instead simply provides an indication regarding a recommended corrective action. In other cases, methods, devices, and systems provided herein can provide a generalized indication of whether the user has a high or low analyte concentration, but not display a specific concentration. In either case, methods and systems provided herein can permit the user to see more specific analyte sensor data, such as specific concentrations, by accessing the remote user-interface device. By limiting the ability of the medication delivery device to display specific analyte sensor data, medication delivery methods and systems provided herein can ensure that the remote user-interface device remains the primary user interface, yet ensure that a user is alerted to safety concerns in a timely manner so that the user can take appropriate corrective action. Additional information that may be displayed on the medication delivery device may include an indication that the amount of medication in the medication delivery device is below a threshold or completely depleted, an indication that an amount of power remaining in a battery in the medication delivery device is below a threshold, an indication that the fluid path for the medication is occluded, or an indication that a message is awaiting the user on the remote user-interface device.

In some cases, methods and systems provided herein can have icons on the medication delivery device that match icons used in the remote user-interface device for communicating alarm or alert conditions. Having matching icons on the medication delivery device and on the remote user-interface device can reinforce the meaning behind these icons as the user uses the system. Additionally, matching icons may provide a user with a more robust explanation of an icon at the remote user-interface device.

In some cases, a remote user-interface device can be adapted to permit the user to enter contextual information regarding the user (e.g., about their condition, about their physical attributes, etc.), and the remote user-interface device can wirelessly communicate the contextual information to a controller in the medication delivery device for use in automating the delivery of medication to the user when in an automated mode. For example, in the case of a person with diabetes (PWD) using an insulin delivery system provided herein, the remote user-interface device can be configured to permit the PWD to enter a meal (e.g., enter an amount of carbohydrates consumed by the PWD). In some cases, a PWD can issue a command for the insulin delivery device to deliver a bolus of insulin for a meal. In some cases, insulin delivery methods and systems provided herein can include a remote user-interface device adapted to permit a user to enter exercise, sickness, menses, other medications (e.g., acetaminophen), exogenous insulin, or any other condition that may impact blood glucose levels or the validity of glucose sensor data. In some cases, medication delivery devices provided herein may not permit the entry of such contextual information, which can reinforce the use of the remote user-interface device as the primary user interface.

Medication delivery devices provided herein can include the controller that determines an amount of medication to deliver, rather than merely being directed to deliver certain amounts of medication by a controller on a remote device, in order to minimize missed or inappropriate medication deliveries due to a faulty wireless connection between components of systems provided herein. Accordingly, in some cases, systems and methods provided herein can have a controller in the medication delivery device be the center of an on-body network and the source of truth such that any inconsistencies or conflicting instructions are resolved based on the controller in the medication delivery device. In some cases, a medication delivery device can include a programmed schedule of medication deliveries or rates and/or other user-specific dosage parameters that determine appropriate dosages of medication. In some cases, the controller in the medication delivery device can update or personalize these schedules and/or dosage parameters over time and send these updated schedules and/or dosage parameters to the remote user-interface device for viewing or use by the user. In some cases, a medication delivery device receiving a command to deliver a bolus of medication can double check the command to see if the dosage is appropriate and/or safe for the user and/or whether the remote user-interface device may have used the wrong schedule or dosage parameters in making a recommendation to the user based on user entered data. In some cases, methods, devices, and systems provided herein may require a user to confirm a desire to deliver a bolus by pushing a button or tapping the medication delivery device under certain conditions. For example, in an insulin delivery system provided herein, a user entering an extremely large meal bolus for that user, or entering multiple bolus amounts within a short period of time, may be required to confirm the bolus by pressing the button or tapping on the housing of the insulin delivery device.

Medication delivery devices provided herein can include a sound emitter adapted to play one or more alarm or alert tones, a vibration motor, and/or one or more indicator lights and/or illuminable icons, where the sound emitter, vibration motor, indicator lights or icons, or any combinations thereof are adapted to indicate whether the medication delivery device is delivering medication, whether a medication delivery rate or amount is being determined at least in part on real-time data from the analyte sensor, whether real-time data is available to the medication delivery device, whether the medication delivery device is low or out of medication, whether the medication delivery device is low on power, and/or whether a message is awaiting the user on the remote user-interface device. In some cases, the medication delivery device can be an insulin pump having a sound emitter, a vibration motor, indicator lights and/or icons, or any combinations thereof and can be adapted to indicate that the user needs to consume a meal and/or that the user should administer a bolus. For example, in some cases, a predictive algorithm in an insulin pump can predict a dangerously low blood glucose level that will not be corrected by suspending insulin delivery, and thus the insulin pump provided herein can indicate to the user that the user should consume carbohydrates. In some cases, an insulin pump provided herein can predict that a maximum administration of basal insulin allowed by the programming of the pump will not return the user to a target blood glucose value or range within a predetermined amount of time, and thus the insulin pump provided herein can indicate that the user should administer a corrective bolus. In some cases, an insulin pump provided herein may use data from a continuous glucose monitor to detect a possible meal that was not accompanied by a bolus, and may indicate that the user should administer a bolus.

In some cases, methods, systems, and devices provided herein can include a button on the medication delivery device, which can be used, in addition to acknowledging/snoozing alarms and alerts, to check the status of the medication delivery device (e.g., check to see if it is delivering medication, a current mode, and see if there are any outstanding messages). For example, if a user wishes to know the current mode of the medication delivery device, the user can double press the button, and an indication light or icon can illuminate to indicate the current mode of operation (e.g., automated mode or non-automated mode). In some cases, additional icons may illuminate or flash to indicate other conditions or messages awaiting the user on the remote user-interface device. For example, if an amount of medication remaining is below an alert threshold level, but not below an alarm threshold level (e.g., at a level where corrective action may be advisable, but not necessary), pressing the button may cause a message icon to light and/or for an insulin depleted or low icon to flash. In some cases, in order for a user to know that all of the lights or illuminable icons are working properly, certain conditions may cause all of the lights to flash on (e.g., an instruction from the remote user-interface device during a systems check process or whenever medication delivery device receives a new supply of medication). Moreover, the lights or illuminable icons can flash when a user acknowledges or snoozes an alarm using the medication delivery device. Additionally or alternatively, a tap detector can be used to detect a desire from the user to see the system status on the medication delivery device. In some cases, a tap detecting device can be an accelerometer.

In some cases, methods, systems, and devices provided herein can provide audible, visual, and/or haptic alarms via an analyte sensor worn by the user, which can be in addition to or instead of the alarms or alerts provided by the medication delivery device.

One or more cases of the present disclosure may include an on-body networked medication-delivery system. Such a system may include an analyte sensor adapted to generate analyte data for a user and wirelessly transmit the analyte data, and a medication delivery device in wireless communication with the analyte sensor. The medication delivery device can include a medication reservoir or a space to receive a medication reservoir, a drive system adapted to meter the administration of medication out of the medication delivery device, and a feedback feature or features to provide audible, visual, or haptic feedback to a user. The medication delivery device can include a controller adapted to change a dosage of medication based at least in part on the analyte sensor data and can be adapted to issue alarm and alert wireless communications based on a detection of an alarm or alert condition, and a tap detector or button adapted to permit the user to check the status of the medication delivery device or to acknowledge the alert or alarm conditions. The on-body networked medication-delivery system can include a remote user-interface device in wireless communication with the medication delivery device. The remote user-interface device can be adapted to receive the alarm and alert wireless communications from the controller and provide an audible, visual, or haptic alarm or alert message to the user and can permit the user to acknowledge an associated alarm or alert condition. In some cases, the remote user-interface device can be adapted to wirelessly communicate each acknowledgement to the controller. In some cases, the controller can be adapted to trigger an audible, visual, or haptic alarm or alert message via the feedback feature to provide audible, visual, or haptic feedback if the controller fails to receive an acknowledgement of the alert or alarm condition within a predetermined period of time after the controller issues the alarm and alert wireless communication.

In one or more methods, systems, or devices of the present disclosure, the system can be a diabetes management system, and the medication delivery device can be an insulin pump, and the analyte sensor can be a continuous glucose monitor.

In one or more methods, systems, or devices, of the present disclosure, the medication delivery device can be a patch pump.

In one or more methods, systems, or devices, of the present disclosure, the medication delivery device can be an insulin pen or pens.

In one or more methods, systems, or devices of the present disclosure, the medication delivery device can include a durable controller and a disposable pump body, each having a housing and being removably connectable. In such cases, the disposable pump body can include at least the medication reservoir or a space to receive a medication reservoir and the durable controller can include at least the feature to provide audible, visual, or haptic feedback, the controller, and the tap detector or button.

In one or more methods, systems, or devices of the present disclosure, the medication delivery device can include a button.

In one or more methods, systems, or devices of the present disclosure, the feature(s) to provide audible, visual, or haptic feedback to a user can include at least one light associated with an icon.

In one or more methods, systems, or devices of the present disclosure, the remote user-interface device can be adapted to present the icon for an alarm or alert condition.

In one or more methods, systems, or devices, of the present disclosure, the at least one light associated with the icon may not illuminate on the housing until the tap detector detects a tap, or the button is pressed, or until the predetermined period of time.

In one or more methods, systems, or devices of the present disclosure, a user can acknowledge an audible, visual, or haptic alarm or alert message provided by a remote user-interface device by tapping the medication delivery device or pressing the button on the medication delivery device even before the medication delivery device triggers the audible, visual, or haptic alarm or alert message via the feature(s) to provide audible, visual, or haptic feedback.

In one or more methods, systems, or devices of the present disclosure, feature(s) to provide audible, visual, or haptic feedback can include a vibration motor adapted to provide haptic feedback, and the controller can be adapted to provide haptic feedback or audible feedback, upon issuing the alarm and alert wireless communications. Additionally, the audible alarm or alert message that can be triggered if the controller fails to receive an acknowledgement of the alert or alarm condition within a predetermined period of time can be louder or longer in duration than the haptic feedback or audible feedback provided when the controller issues the alarm and alert wireless communications.

In one or more methods, systems, or devices of the present disclosure, the predetermined period of time can be at least 30 seconds and no greater than 1 hour, between 1 minute and 30 minutes, between 3 minutes and 20 minutes, or between 5 minutes and 15 minutes, and the predetermined period of time for an alarm or alert condition can depend on the alarm or alert condition.

In one or more methods, systems, or devices of the present disclosure, an acknowledgement of an alarm or alert may quiet audible or haptic feedback for the alarm or alert condition for a predetermined snooze period of time, and the controller can be adapted to issue new alarm and alert wireless communications after the predetermined snooze period of time if the alarm or alert condition is still detected as being present.

In one or more methods, systems, or devices of the present disclosure, the remote user-interface device can be adapted to present the user with troubleshooting instructions using text, audio, or video to remove the alarm or alert condition, and the medication delivery device may not present any troubleshooting instructions using text, audio, or video.

In one or more methods, systems, or devices of the present disclosure, the medication delivery device can include a housing that contains a non-rechargeable, non-replaceable battery.

In one or more methods, systems, or devices of the present disclosure, the remote user-interface device can be adapted to allow a user to send instructions to the medication delivery device using the remote user-interface device, and the remote user-interface device can prompt the user to confirm the instructions by pressing the button or tapping the controller under certain conditions.

In one or more methods, systems, or devices of the present disclosure, the controller can be adapted to require a user to confirm a bolus delivery by pressing the button or tapping the controller if the dosage is determined by the controller to be unusual based on typical dosage amounts administered by the user, based on the timing the dosage or the timing of a previous dosage, or based on a prediction of how the dosage will change analyte levels for the user.

In one or more methods, systems, or devices of the present disclosure, the medication delivery device can include one or more icons, and one or more lights associated with those one or more icons, indicating whether the medication is being delivered based on the analyte sensor or not or whether there is an error with the analyte sensor.

In one or more methods, systems, or devices of the present disclosure, the medication delivery device can include one or more icons, and one or more lights associated with those one or more icons, indicating that an amount of medication in the medication delivery device is below a threshold level.

In one or more methods, systems, or devices of the present disclosure, the medication delivery device can include one or more icons, and one or more lights associated with those one or more icons, indicating that the user should administer more medication or consume carbohydrates.

In one or more methods, systems, or devices of the present disclosure, the medication delivery device can include one or more icons, and one or more lights associated with those one or more icons, indicating that a more detailed message for the user is awaiting the user on the remote user-interface device.

One or more cases of the present disclosure can include a method for issuing alarms and alerts in an on-body networked diabetes management system. The method can include receiving glucose sensor data from a continuous glucose monitor, and determining a dosage of insulin delivery based at least in part on the glucose sensor data. The method can include detecting an alarm or alert condition, and sending a wireless communication regarding the alarm or alert condition to a remote user-interface device. The method can additionally include triggering an audible, visual, or haptic alarm or alert on the insulin delivery device if the insulin delivery device does not receive an acknowledgement of the alarm or alert condition within a predetermined period of time.

In one or more methods, systems, or devices of the present disclosure, the user can acknowledge the alarm by pressing a button on the insulin delivery device or by tapping the insulin delivery device and by interacting with the remote user-interface device, and the insulin delivery device can receive an acknowledgement of the alarm or alert condition as part of a wireless communication from the remote user-interface device.

In one or more methods, systems, or devices of the present disclosure, such a method can include triggering audible or haptic feedback of the insulin delivery device when sending the wireless communication regarding the alarm or alert condition to the remote user-interface device, and the audible, visual, or haptic alarm or alert on the insulin delivery device after the predetermined period of time can be louder or longer in duration than the feedback initiated when sending the wireless communication.

In one or more methods, systems, or devices of the present disclosure, such a method can include stopping the audible, visual, or haptic alarm or alert on the insulin delivery device when a button on the insulin delivery device is pressed.

In one or more methods, systems, or devices of the present disclosure, the button must be pressed at least twice during a predetermined period of time or according to a predetermined pattern for the audible, visual, or haptic alarm or alert to be stopped.

In one or more methods, systems, or devices of the present disclosure, stopping the audible, visual, or haptic alarm or alert on the insulin delivery device can prevent the triggering of any audible, visual, or haptic alarms or alerts regarding that alarm or alert condition or the sending of any wireless communication regarding the alarm or alert condition for a predetermined period of time. Additionally, one or more methods or processes of the present disclosure can repeat after the predetermined period of time if the alarm or alert condition is present after the predetermined period of time.

In one or more methods, systems, or devices of the present disclosure, the alarm or alert condition can indicate a change from a first mode of operation to a second mode of operation.

In one or more methods, systems, or devices of the present disclosure, the alarm or alert condition can be an indication of an amount of insulin remaining in the insulin delivery device being below a threshold level.

In one or more methods, systems, or devices of the present disclosure, the alarm or alert condition can be an indication of a low glucose condition or a high glucose condition.

In one or more methods, systems, or devices of the present disclosure, the audible, visual, haptic alarm or alert on the insulin delivery device can include the illumination of an icon or next to an icon indicating that the user should eat or should administer insulin.

In one or more methods, systems, or devices of the present disclosure, the alarm or alert condition can be a notice that the continuous glucose monitor is not working, not in range, or not reliable.

In one or more methods, systems, or devices of the present disclosure, the alarm or alert condition can be a notice about a possible occlusion, a possible air bubble, a possible missed meal announcement, a possible need to change an infusion set, a possible need to calibrate a CGM, a possible need to replace the CGM, or a possible need to check ketone levels, and the audible, visual, haptic alarm or alert on the insulin delivery device can include the illumination of an icon or next to an icon indicating that the user should check the remote user-interface device for information about the alert.

One or more cases of the present disclosure can include an insulin delivery device adapted for wireless communication with a continuous glucose monitor and a remote user-interface device. The insulin delivery device can include an insulin reservoir or a space to receive an insulin reservoir, and a drive system adapted to meter the administration of insulin out of the insulin delivery device. The insulin delivery device can include a wireless transmitter and receiver adapted to send and receive wireless communications from at least a continuous glucose monitor and a remote user-interface device, and a controller adapted to change a dosage of medication based at least in part on data from the continuous glucose monitor and adapted to issue alarm and alert wireless communications based on a detection of an alarm or alert condition. The insulin delivery device can additionally include a housing containing at least the controller and the wireless transmitter and receiver, a tap detector within the housing or a button on the housing adapted to permit the user to check the status of the insulin delivery device or to acknowledge alert or alarm conditions, and one or more lights adapted to illuminate icons or adjacent to icons on the housing indicating a mode of operation of the insulin delivery device and whether insulin is being delivered to the user.

In one or more methods, systems, or devices of the present disclosure, an insulin delivery device can include one or more lights or another visual or audio cue adapted to illuminate icons or adjacent to icons on the housing indicating that a message is awaiting the user on the remote user-interface device.

In one or more methods, systems, or devices of the present disclosure, an insulin delivery device can include one or more lights adapted to illuminate icons or adjacent to icons on the housing indicating that the user has a blood glucose condition requiring the consumption of carbohydrates or the administration of additional insulin.

In one or more methods, systems, or devices of the present disclosure, the user cannot administer additional insulin using the insulin delivery device without accessing the remote user-interface device.

In one or more methods, systems, or devices of the present disclosure, the controller can be adapted to evaluate whether a wireless communication from a remote user-interface device is within one or more predefined parameters.

In one or more methods, systems, or devices of the present disclosure, the controller can be adapted to send a wireless communication to the remote user-interface device indicating that a bolus is outside of one or more predefined parameters, or indicating the user must confirm the bolus on the insulin delivery device by tapping or pressing the button.

In one or more methods, systems, or devices of the present disclosure, an insulin delivery device can include one or more lights adapted to illuminate icons or adjacent to icons on the housing indicating that there is a problem with the data being received, or a lack of data being received, from the continuous glucose monitor.

One or more cases of the present disclosure can include a medication delivery system that includes a medication delivery device and a remote user-interface device, where the medication delivery device and the remote user-interface device can be in wireless communication. The medication delivery device can be adapted to automatically administer medication according to a programmed rate, a programmed schedule, or based on analyte sensor data without user input. The remote user-interface device can be adapted to receive user commands for the medication delivery device to administer additional doses of medication, adjust the programmed delivery rate or schedule, or adjust an algorithm that determines a dosage based on the analyte sensor data. Additionally, both the remote user-interface device and the medication delivery device can be adapted to provide audible, visual, or haptic feedback to issue an alarm or alert regarding the ability of the medication delivery device to deliver medication. The medication delivery device can be adapted to detect a condition that prevents the delivery of medication and send an alarm wireless communication to the remote user-interface device regarding the condition. The remote user-interface device can issue an audible, visual, or haptic alarm when the alarm wireless communication is received, and can provide a feature for the user to acknowledge the alarm. The remote user-interface device can send an acknowledgement wireless communication to the medication delivery device upon the user acknowledging the alarm, and the medication delivery device can be adapted to issue an audible, visual, or haptic alarm after a predetermined period of time after the alarm wireless communication is sent unless the medication delivery device receives the acknowledgement wireless communication during the predetermined period of time.

In one or more methods, systems, or devices of the present disclosure, the medication delivery device can include a feature to receive a user's acknowledgement an audible, visual, or haptic alarm to silence the alarm.

In one or more methods, systems, or devices of the present disclosure, the medication delivery device can include an insulin infusion pump, the medication can be insulin, and the remote user-interface device can be a smartphone.

In one or more methods, systems, or devices of the present disclosure, such a system can include a continuous glucose monitor in wireless communication with the insulin infusion pump, and the insulin infusion pump can deliver different amounts or rates of insulin based on glucose data from the continuous glucose monitor.

In one or more methods, systems, or devices of the present disclosure, the insulin infusion pump may not be adapted to display specific concentrations of the glucose data, but can be adapted to send glucose data wireless communications to the smartphone, and the smartphone can be adapted to display specific concentrations of the glucose data.

In one or more methods, systems, or devices of the present disclosure, the insulin infusion pump can be adapted to illuminate one or more icons, or a light next to one or more icons, that indicate that the user is in or expected to experience hypoglycemic state or a hyperglycemic state, that indicate that the user should administer more insulin, or that indicate that the user should consume food, and that light can become illuminated when the insulin infusion pump issues an issue an audible, visual, or haptic alarm or when the user acknowledges the alarm on the insulin infusion pump.

In one or more methods, systems, or devices of the present disclosure, the insulin infusion pump can be adapted to illuminate one or more icons, or a light next to one or more icons, that indicate that the user is in out of insulin, wherein the light becomes illuminated when the insulin infusion pump issues an issue an audible, visual, or haptic alarm or when the user acknowledges the alarm on the insulin infusion pump.

The details of one or more implementations of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1A is a perspective view of a first example medication delivery system including a module medication delivery device, at least one analyte sensor, and a remote user-interface device.

FIG. 1B is a perspective view of a second example medication delivery system including a patch pump-type medication delivery device, at least one sensor, and a remote user-interface device.

FIG. 2 is an example view of a module medication delivery device showing an example medication delivery device user interface.

FIG. 3 depicts an example user interface landing home screen for a remote user-interface device.

FIG. 4 is an infographic regarding alarms, alerts, and other notices used in a diabetes management system.

FIGS. 5A-5C depict example alarms for a lack of insulin flowing in an insulin delivery system provided herein. FIG. 5A depicts a visual indication for the alarm on an insulin delivery device. FIGS. 5B and 5C depict a visual indication for the alarm on a remote user-interface device.

FIG. 6 depicts an example of how the alarms of FIGS. 5A-5C can progress on the remote user-interface device to a tutorial on how to resolve the alarm condition.

FIG. 7A depicts an example of how an alert may appear on a remote user-interface device.

FIG. 7B depicts an example of how a notification may appear on a remote user-interface device.

FIGS. 8A and 8B depict examples of how multiple alarms may appear on a remote user-interface device.

FIG. 9A depicts an example progression of alarm notifications for a lack of insulin flowing if the user acknowledges the alarm condition within a predetermined period of time.

FIG. 9B depicts an example progression of alarm notifications for a lack of insulin flowing if the user fails to acknowledge the alarm condition within a predetermined period of time.

FIG. 10A depicts an example progression of an alert notification regarding battery or insulin levels if the user acknowledges the alert condition within a predetermined period of time.

FIG. 10B depicts an example progression of an alert notification regarding battery or insulin levels if the user fails to acknowledge the alarm condition within a predetermined period of time.

FIG. 11A depicts an example progression of an alert notification regarding a first blood glucose event if the user acknowledges the alert condition within a predetermined period of time.

FIG. 11B depicts an example progression of an alert notification regarding a first blood glucose event if the user fails to acknowledge the alarm condition within a predetermined period of time.

FIG. 12A depicts an example progression of an alert notification regarding a second blood glucose event if the user acknowledges the alert condition within a predetermined period of time.

FIG. 12B depicts an example progression of an alert notification regarding a second blood glucose event if the user fails to acknowledge the alarm condition within a predetermined period of time.

FIG. 13A depicts an example progression of an alert notification regarding a CGM event if the user acknowledges the alert condition within a predetermined period of time.

FIG. 13B depicts an example progression of an alert notification regarding a CGM event if the user fails to acknowledge the alarm condition within a predetermined period of time.

FIG. 14A depicts an example progression of an alert notification regarding a mode change event if the user acknowledges the alert condition within a predetermined period of time.

FIG. 14B depicts an example progression of an alert notification regarding a mode change event if the user fails to acknowledge the alarm condition within a predetermined period of time.

FIGS. 15A-15C illustrate how example notification lights on an automated medication infusion pump can inform a user about the status of the medication delivery system.

FIGS. 16A and 16B illustrate example alarms and how a user can snooze the alarms.

Like reference symbols in the various drawings may indicate like elements.

### DETAILED DESCRIPTION

Methods, devices, and systems provided herein can be used to deliver any appropriate medication for the treatment of any appropriate disease or condition. The embodiments described below relate to an insulin delivery system for the management of diabetes, however, the delivery of other types of medications for other diseases are also contemplated. For example, in addition to diabetes, methods, devices, and systems provided herein can be used to treat unresponsive infections, cancer, cancer-related pain, chronic pain, gastrointestinal diseases or disorders, congestive heart failure, hemophilia, immune deficiencies, multiple sclerosis, and rheumatoid arthritis. In some cases, methods, devices, and systems provided herein can use analyte sensor data to automate the delivery of medication. Although the example embodiments described below are specific to an insulin delivery device adapted to automate basal insulin deliveries based on data from a continuous glucose monitor, medication delivery systems that do not include and/or consider data from an analyte sensor are also contemplated.

As used herein, the term “substantially” in reference to a given parameter feature(s) and includes to a degree that one skilled in the art would understand that the given parameter, property, or condition is met with a small degree of variance, such as within acceptable manufacturing tolerances. For example, a parameter that is substantially met may be at least about 90% met, at least about 95% met, or even at least about 99% met.

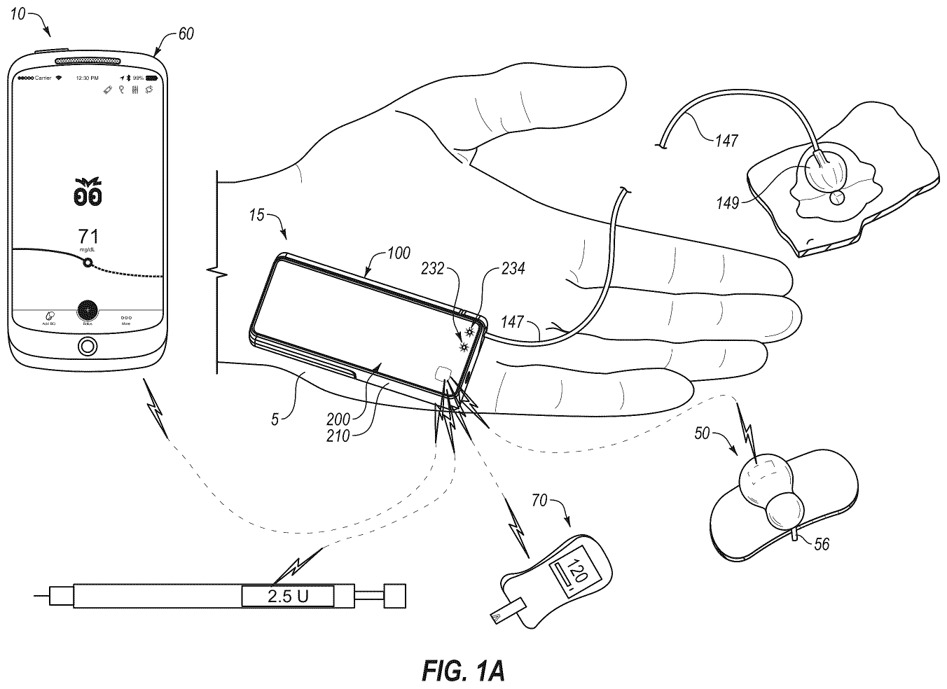

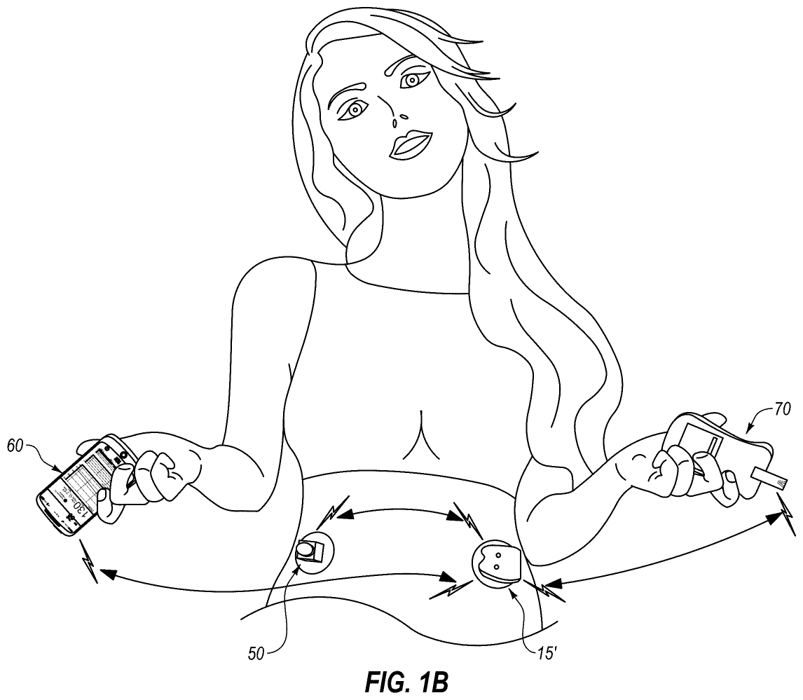

FIG. 1A depicts an example medication delivery system provided herein, which includes at least a medication delivery device 15 and a remote user-interface device 10. As shown, the medication delivery device 15 is an insulin delivery device, more specifically an insulin infusion pump. As shown, the medication delivery device 15 can be sized to fit within an adult human's hand 5. In some embodiments, the medication delivery system depicted in FIG. 1A may be similar to or the same as that disclosed in U.S. Patent Application Publication No. US 2017/0203037 A1, published Jul. 20, 2017, the disclosure of which is hereby incorporated herein in its entirety by this reference.

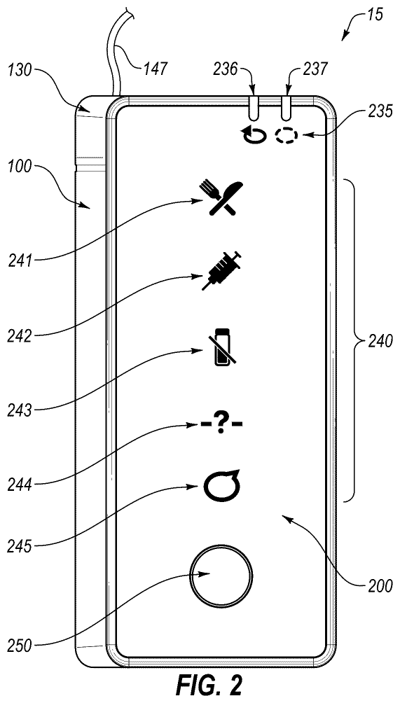

FIG. 1A further depicts analyte sensor 50, which are shown as both being in wireless communication with the medication delivery device 15. Analyte sensor 50 can be a continuous glucose monitor (CGM, and may be referred to as CGM 50) adapted to have a sensor probe 56 sit subcutaneously on a user's skin and provide regular (e.g., every 1 minute, every 3 minutes, every 5 minutes, every 10 minutes, every 30 minutes, or at some interval in between) or irregular (e.g., variable depending on one or more previous blood glucose readings) blood glucose readings. The medication delivery device 15 can then use the data from CGM 50 to alter medication dosages or delivery rates. Although the incorporated by reference U.S. Patent Application Publication No. US 2017/0203037 A1 describes certain techniques for changing between basal rates of 0×, 1×, and 2× (and optionally 3× or other multipliers) of a baseline basal rate, other techniques for using CGM to automate basal insulin delivery rates or provide microboluses of insulin to a user are contemplated and known in the art. In some cases, the medication delivery device 15 can use the CGM data in a proportional-integral (PI) controller, a derivative controller, a proportional-integral-derivative (PID) controller, a model predictive controller, etc. Additionally, as described in the incorporated by reference U.S. Patent Application Publication No. US 2017/0203037 A1, the medication delivery device 15 can have multiple modes, including an automated mode and a non-automated mode (e.g., a personalized mode), which can be entered and exited based on the availability of actionable CGM data (and optionally other conditions) and/or one or more user preferences. Accordingly, the medication delivery device 15 can include indicator lights 232 and 234, which can be used to indicate a mode of operation, and optionally certain error conditions. Additional details about possible indicator lights and arrangements are described below in relationship to FIGS. 2-15.

FIG. 1B depicts a medication delivery system according to a second embodiment where a medication delivery device is a patch pump 15′, which also can be used to deliver insulin in an insulin delivery system and can use CGM data from CGM 50 to automate insulin dosages or rates. As shown, patch pump 15′ also includes indicator lights. Although FIGS. 2-15 show a medication delivery device user interface on a medication delivery device similar to that of FIG. 1A, the features shown and described below are equally applicable to patch pumps or other suitable delivery devices. Also as shown, patch pump 15′ can include a wireless receiver and transmitter to send and receive wireless communications from a remote user-interface device 60, the CGM 50, and a blood glucose meter 70, and can include a controller adapted to automate insulin based at least in part on CGM data using any appropriate control algorithm.

FIG. 2 depicts an example user interface for the medication delivery device 15, specifically for an insulin infusion pump. In some cases, the medication delivery device 15 can be a modular medication delivery device including a disposable pump body 100 and a durable controller 200, as described in the incorporated by reference U.S. Patent Application Publication No. US 2017/0203037 A1. However, this user interface can be applied to unitary medication delivery devices, as well as to patch pumps (such as those shown in FIG. 1B) and any other suitable medication delivery device, particularly those used to deliver insulin. As shown, a front face of medication delivery device 15 can include a button 250. In some cases, medication delivery devices provided herein can include only one button. In some cases, medication delivery devices provided herein can have a plurality of buttons. In some cases, medication delivery devices provided herein can only have buttons capable of snoozing, quieting, or acknowledging alarms, alerts, or notifications and for checking the system status.

The user interface can additionally include a plurality of indicator lights and/or illuminable icons. As shown, indicator lights 236 and 237 can be positioned adjacent to icons 235. As shown, lights and icons 235-237 can inform the user whether the user is in an automated (e.g., closed-loop, open-loop, partially closed-loop, etc.) mode or a non-automated (e.g., personalized) mode. The illumination of light 236 indicates an automated mode and the illumination of light 237 indicates a non-automated mode. In some cases, additional mode lights can be used to indicate other modes. In some cases, a single light can be used to indicate a mode (e.g., a color, flashing pattern, or other light characteristic) can be used to indicate the current mode. In some cases, a plurality of alarm or alert illuminable icons 240 can be positioned on the housing to indicate the need for the user to take certain actions. Although the mode lights 236 and 237 are depicted as being adjacent to icons and icons 241-245 being indicated as being illuminable, the opposite is also a contemplated design, and all icons could have an adjacent light or all icons could be illuminable. As shown, illuminable icon 241 is illuminated, while the other icons 242-245 are not illuminated. Illuminable icon 241 represents a need to cat, illuminable icon 242 indicates a need to take insulin, illuminable icon 243 indicates a depletion of insulin in the medication delivery device 15, illuminable icon 244 indicates an error with CGM data, and illuminable icon 245 indicates that a message awaits the user on the remote user-interface device. Although specific icons are depicted, other icons are also contemplated.

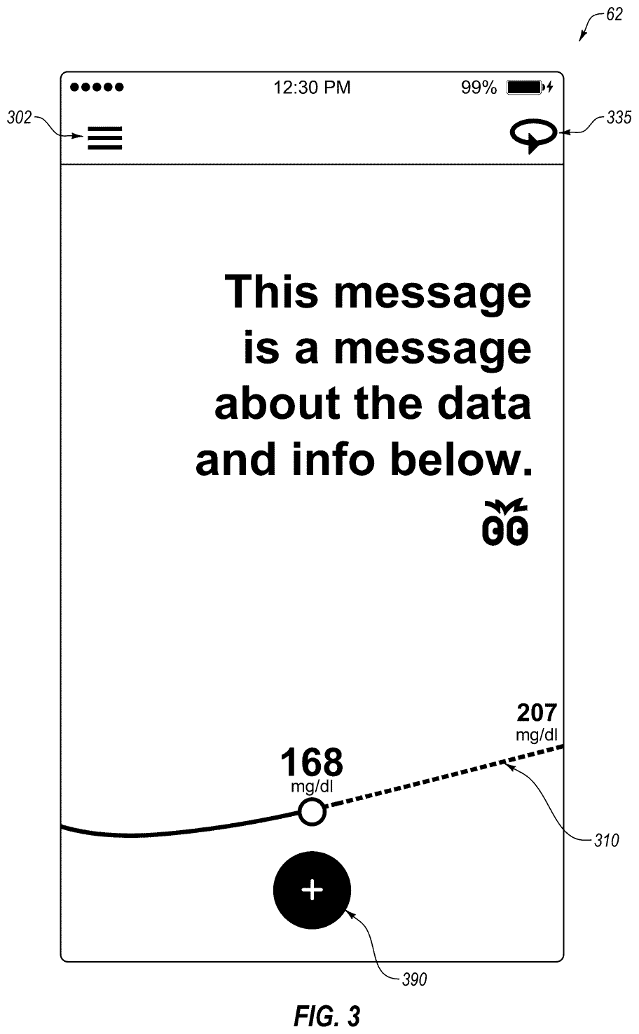

FIG. 3 depicts an example home landing screen 62 for remote user-interface device 10. As described in the incorporated by reference U.S. Patent Application Publication No. US 2017/0203037 A1 (using a different name), remote user-interface device 10 can be a smartphone or any other suitable remote device having a suitable display and robust data entry capabilities (e.g., a PDA, a tablet computer, a music-playing device). In some cases, the remote user-interface device 10 has an application stored in memory to execute the user interfaces and user experience provided herein. As shown, the remote user interface 60 includes a navigation menu 302, a mode indicator icon 335, a message, blood glucose data display 310 illustrated with a blood glucose value, a blood glucose trend line, and a blood glucose prediction, and a bolus button/user-selectable icon 390. As shown, mode indicator icon 335 can resemble or match an icon 235 on the medication delivery device housing 210, which can reinforce the meaning of the icon. In some cases, if a user taps on the icon in the remote user interface 60, a message can appear explaining the meaning of the icon. If a user taps on the blood glucose data display 310, a message can appear providing an explanation of the display and/or bring the user to a more specific display of blood glucose data and/or insulin delivery data.

Examples of more detailed chart displays of blood glucose data aligned with insulin delivery data are depicted and described in the incorporated by reference U.S. Patent Application Publication No. US 2017/0203037 A1. Navigation menu 302 can be pressed or tapped by the user to access other functions of the remote user-interface device, such as instructional videos on performing certain tasks, entering other contextual information, setting up personal preferences, etc. Bolus button/user-selectable icon 390 can be prominent on the home screen because it can be one of the most important functionalities of the remote user-interface device 10 with respect to controlling the operation of the medication delivery device 15. An additional important function of the remote user-interface device 10 being to provide the user with actionable information regarding alarms, alerts, and other notifications useful for managing/treating diabetes. In some cases, pressing the bolus button 390 can bring the user to a bolus calculator that helps the user determine a bolus based on entered food information, blood glucose data, stored personal dosage parameters (e.g., an insulin sensitivity factor and a carbohydrate-to-insulin ratio), and an estimation of unacted insulin delivered (e.g., insulin on board (IOB)).

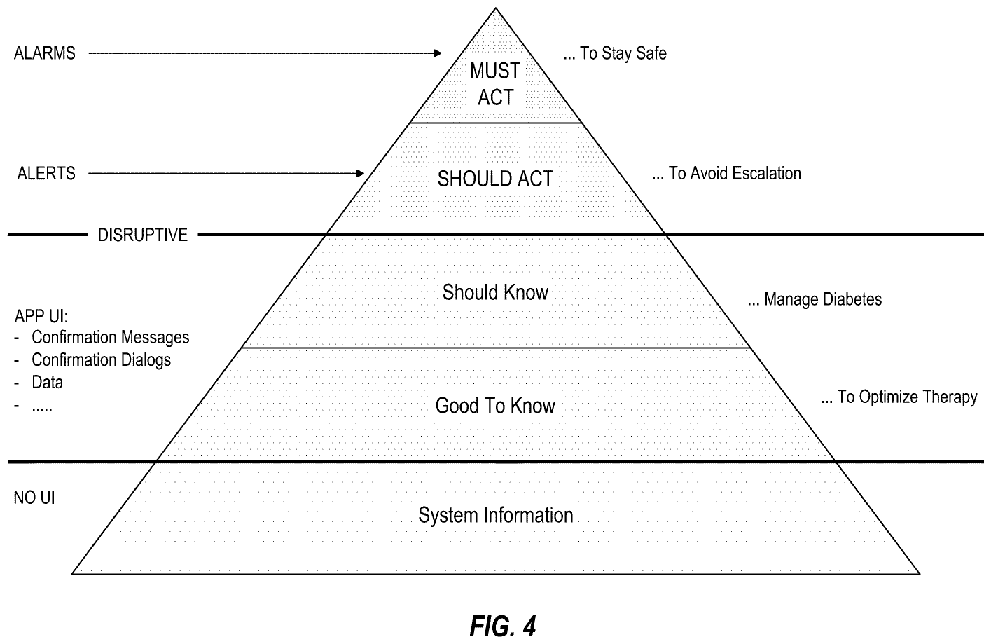

FIG. 4 depicts an infographic depicting conceptually a distinction between alarms, alerts, and notifications, as those terms are used in the present disclosure. In some cases, a notification regarding a condition, if not acted upon by a user, can result in that condition triggering an alert or an alarm. In general, an alarm condition is something that requires a user's attention to stay safe, or in other words, a condition that could result in personal injury or other health complications if the condition is not addressed. An example alarm might be triggered by a severe hypoglycemic event. An alarm event might also be triggered if the medication delivery device has a nearly dead battery or is totally depleted of insulin or if there is an occlusion in an infusion catheter 147. In many cases, an alarm condition may typically be avoided if a user takes action based on the triggering of alerts. An alert condition is something that could lead to an alarm condition, or that needs attention. However, an alert condition does not have the same urgency or immediacy as an alarm condition. For example, a predicted hypoglycemic event may trigger a potential hypoglycemic event alert using different threshold criteria than the severe hypoglycemic event condition so that a user can take corrective action before blood glucose levels drop to a level triggering an alarm event. Other possible alerts may include hyperglycemic events, a failure to receive actionable CGM data, a need to calibrate the CGM, an amount of insulin being below a higher alert threshold, and/or a need to conduct system maintenance or replace system components based on their recommended user life. Alarms and alerts should be disruptive to the user because the user should or must act to remain safe, thus methods, systems, and devices provided herein ensure that a user receives prompt notice of these alarm or alert conditions even if the remote user-interface device is not immediately available to the user. The non-disruptive notifications, however, are things that a user can use to better manage or optimize their treatment of diabetes, but are not urgent, thus they can remain accessible upon demand by the user on the remote user-interface device. Such information might be data about past, current, or predicted blood glucose values that are in a safe range, past and current insulin doses and basal delivery rates, previously entered meal sizes, etc. Additionally, there is system information unrelated to the treatment of diabetes, which can be hidden from the user.

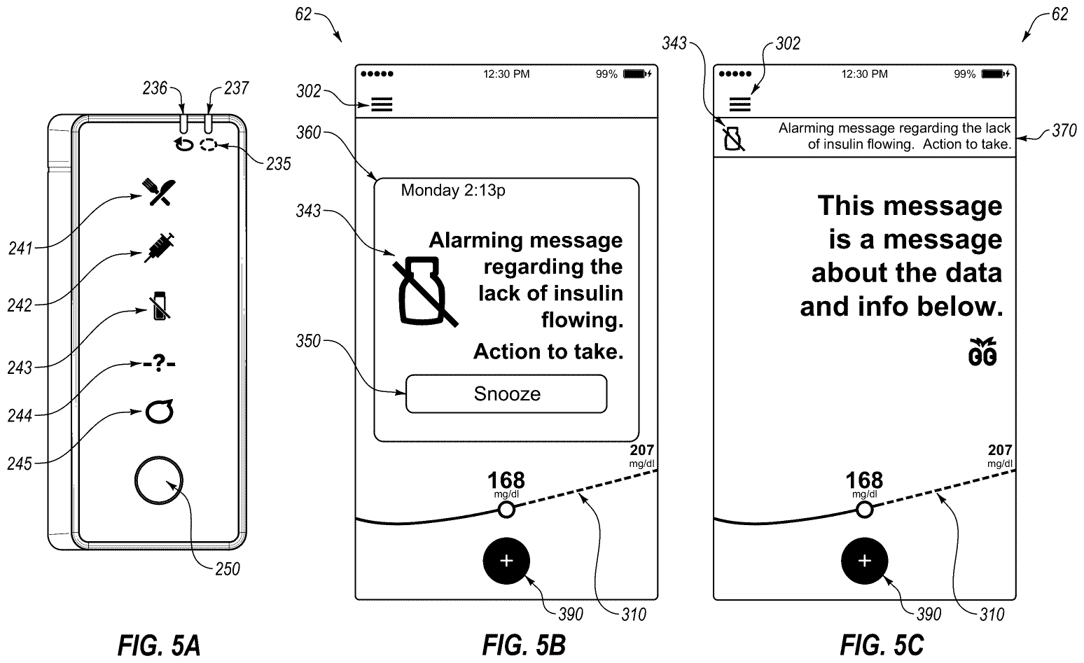

FIGS. 5A, 5B, and 5C illustrate how an alarm condition related to a lack of insulin flowing can be displayed on the medication delivery device 15 (FIG. 5A) and on the remote user-interface device (FIGS. 5B and 5C). In each case, the visual indicators shown can be accompanied with an audible alarm tone and/or haptic feedback. As shown, the icon 243 for this alarm condition on the medication delivery device 15 matches an icon 343 displayed on the remote user-interface device (FIGS. 5B and 5C). As shown in FIG. 5B, an alarm notification box 360 can pop up when the user accesses an app on a smartphone. By pressing snooze button 350, the user can stop the audible alarm tone and/or haptic feedback. Similarly, by pressing button 250 on medication delivery device 15, any audible alarm tone sounding from the medication delivery device 15 can be stopped. In some cases, pressing button 250 can result in the medication delivery device 15 sending a wireless communication to remote user-interface device 10 to stop an audible alarm tones or haptic feedback, and vice-versa regarding using button 350 to stop alarm tones and haptic feedback on the medication delivery device. Regarding FIG. 5A, illuminating icon 243 can in some case be illuminated as soon as audible and/or haptic alarming occurs on the medication delivery device 15, or can be illuminated at the point in time when the user presses button 250 to silence the audible/haptic alarm to show the user the reason for the alarm. In either case, illuminating icon 243 can remain illuminated after snoozing the alarm for at least a predetermined period of time (e.g., between 1 second and 5 minutes, or between 2 seconds and 1 minute, or between 5 seconds and 30 seconds). Referring back to FIG. 5B, alarm notification box 360 can appear over the home landing screen of the remote user-interface device, but can appear over other screens of the user interface.

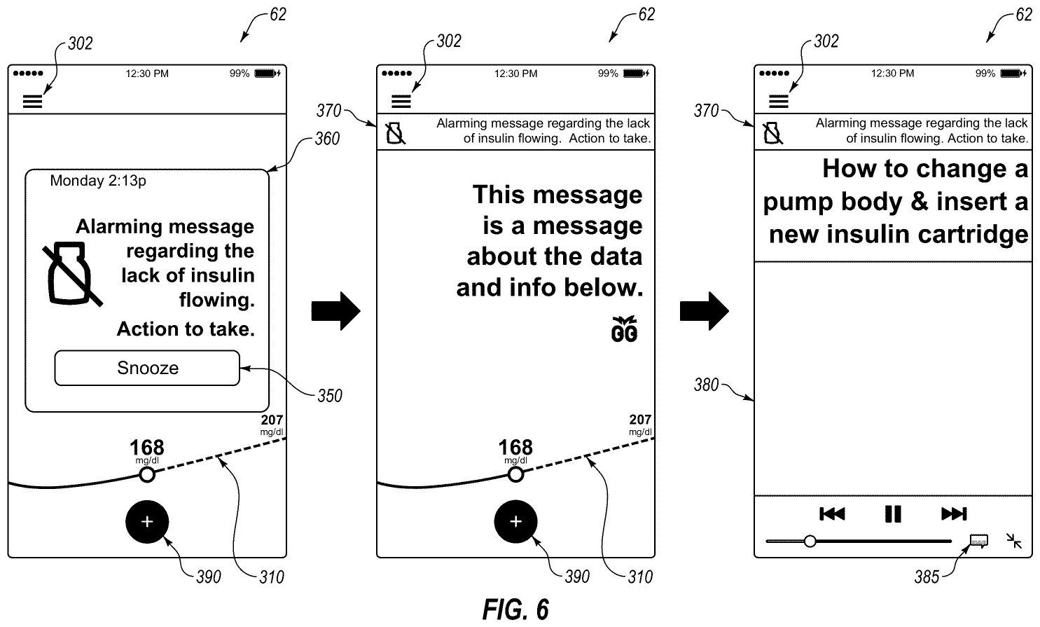

As shown in FIG. 6, after an alarm is snoozed by pressing button 350 on the user interface of remote user-interface device 10, the alarm notification box may become an alarm notification banner, still retaining the same icon 343. In some cases, a user may click on this banner 370 to find out additional information about how to resolve the alarm condition (as indicated in FIG. 6). FIG. 6 shows the flow of where a user may push or tap banner 370 to be taken to a screen that includes a troubleshooting view 380, which may optionally include a chat icon 385 to allow the user to live chat with an expert on using the medication delivery system to help the user troubleshoot the problem.

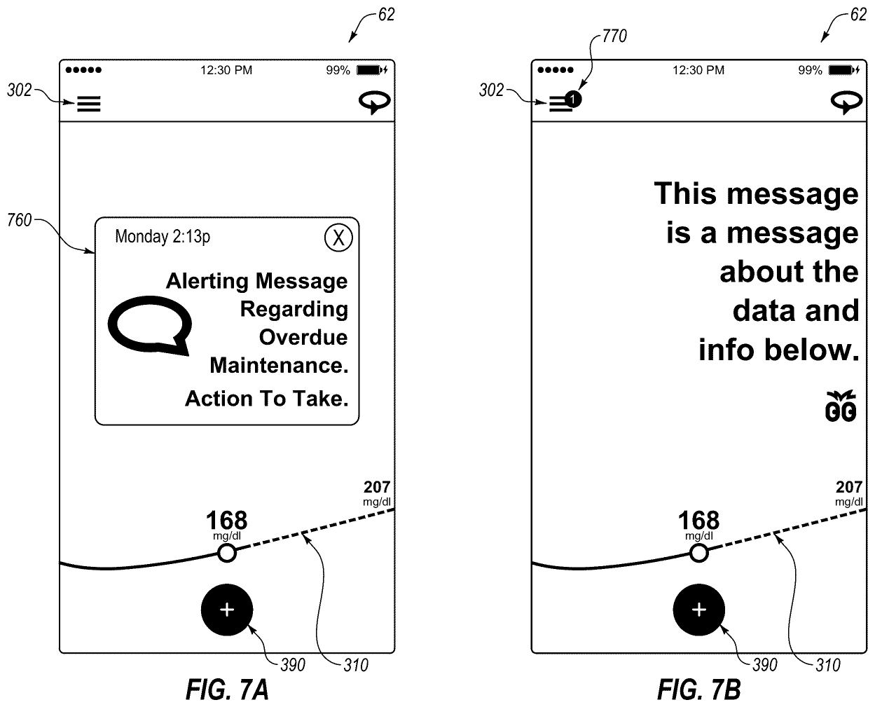

FIG. 7A depicts an example of how an alert may appear on a remote user-interface device. As shown, an alert may appear as an alert box 760, which may be acknowledged by the user, similar to the way that an alarm can be acknowledged by a user. As shown, the icon in alert box 760 mirrors illuminable icon 245, which can indicate that there is a message available for the user on the remote user-interface device. By having icons mirrored between the remote user-interface and the medication delivery device, the messages associated with each of icons 241-245 can be reinforced in the user's mind. As shown, the message can be about an overdue maintenance task, such as changing any infusion set 149.

FIG. 7B depicts an example of how a notification may appear on a remote user-interface device. Because notifications do not require immediate action, they can appear as a notation 770 on the navigation menu 302 to indicate to the user that a non-urgent message is available for them. In some cases, a snoozed alert can become a notification message available as a notation 770 until such time as an alert snooze time period runs out or the alert condition escalates to a different alarm condition. In some cases, certain alert conditions can become banners after being snoozed.

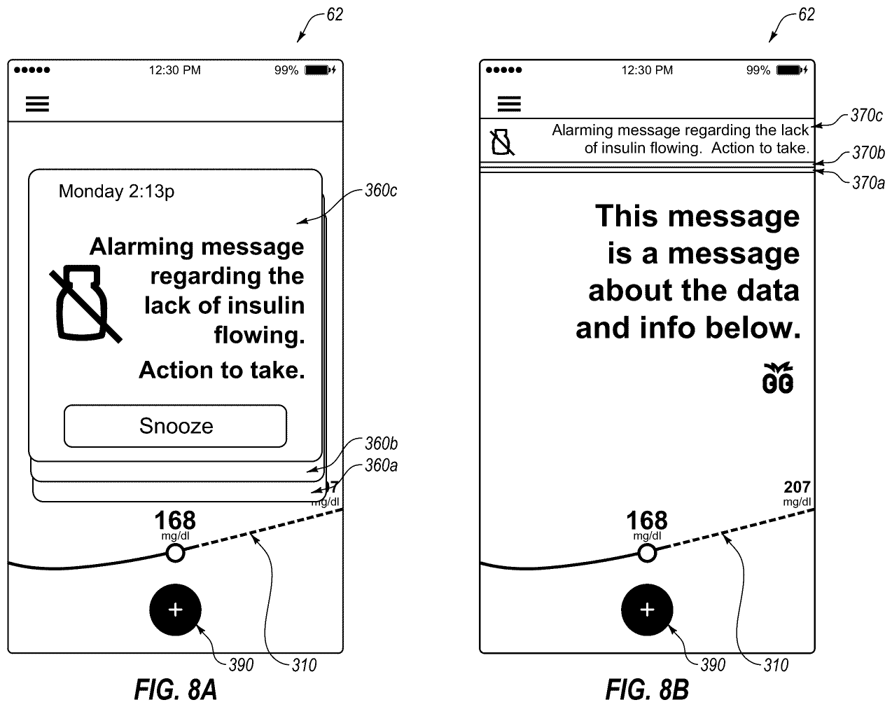

FIGS. 8A and 8B depict examples of how multiple alarms may appear on a remote user-interface device. FIG. 8A depicts multiple alarm boxes 360a, 360b, and 360c stacked on top of each other. As each is cleared, the box underneath is revealed, and each becomes a banner 370a, 370b, 370c, each stacked on top of each other until the user resolves the alarm condition. In some cases, the most recently triggered alarm is stacked on top. In some cases, alarm conditions can have an order of priority and the most urgent alarm condition can be stacked on top. In some cases, alert boxes 760 can be in a stack of boxes. In some cases, alarm and alert boxes can be distinguished by color and/or size. In some cases, the alert boxes 760 may be staggered along the screen (e.g., vertically staggered) such that at least a portion (e.g., an entirety) of each alert box 760 is visible.

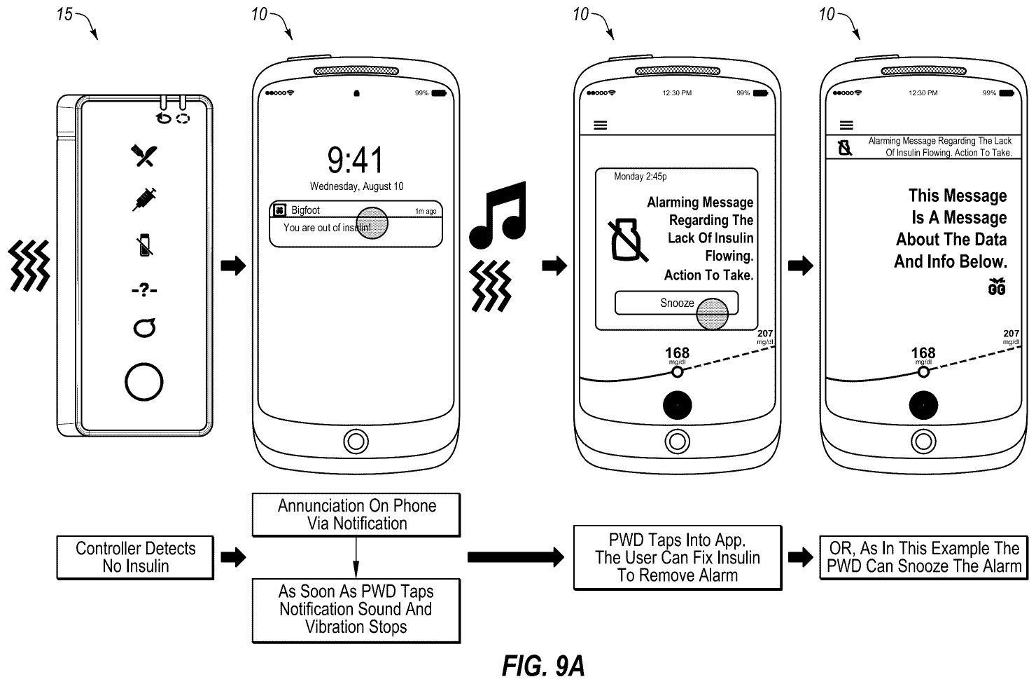

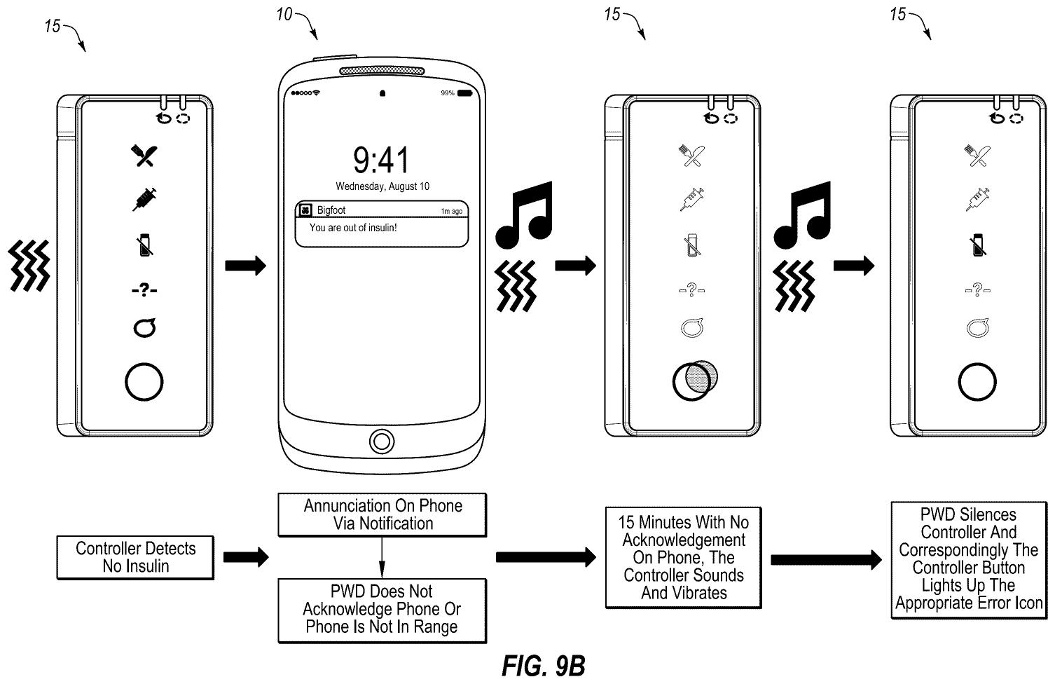

FIG. 9A depicts an example progression of alarm notifications for a lack of insulin flowing if the user acknowledges the alarm condition within a predetermined period of time (where user clicks or acknowledgements are indicated with a circle). As shown, the medication delivery device 15 first detects that there is no insulin or a blockage of insulin and then sends a wireless message to remote user-interface device 10. In some cases, medication delivery device 15 can vibrate upon detecting the alarm condition and/or upon sending the wireless communication. This vibration may be short in duration, and be present in order to let the user know that a notification from the remote user-interface device 10 (e.g., a smartphone, such as an iPHONE®) is from the medication delivery device. After receiving the wireless communication, a message (e.g., an iOS message) can appear on a smartphone version of a remote user-interface device along with sound and/or vibration, assuming that the user permits such notices, vibrations, or sounds for the app of the medication delivery system. The user can then select the message to be brought to the app, where the user can snooze the message and click on the banner to learn how to resolve the issue. In some cases, the user will know how to resolve the issue from experience and not need to click on the banner to resolve the alarm condition.

FIG. 9B depicts an example progression of alarm notifications for a lack of insulin flowing if the user fails to acknowledge the alarm condition within a predetermined period of time. This may be due to the smartphone being out of battery, left at home, or having settings on the smartphone that do not allow alarms to be announced. Regardless, if the medication delivery device 15 fails to receive an acknowledgement within a predetermined period of time, which may depend on the type of alarm condition, the medication delivery device can start to vibrate and sound an alarm tone, until the user presses the button to quiet the alarm sound and/or vibration, upon which an icon 243 will illuminate to tell the user that the alarm condition relates to the supply of insulin. In such a situation, an experienced user of the system may know to check to see if there is insulin remaining in the medication delivery device and/or to change out the insulin cartridge for a new cartridge and/or to check for occlusions. In some cases, a user will know to retrieve the remote user-interface device for help in determining how to resolve the alarm condition. After a predetermined period of time (e.g., between 5 seconds and 30 seconds) illuminated icon 243 may be turned off, but the user can again press the button to check the status of the medication delivery device. Additionally of note, neither of the mode lights 236 or 237 is illuminated, indicating that insulin is not being delivered. The alarm on the pump can have a greater duration and/or volume than any sound/vibration made on pump when pump first sends the wireless communication to the smartphone.

While FIGS. 9A-14B illustrate a smartphone as the remote user-interface device, it will be appreciated that any device with any notification scheme with locked devices, unlocked devices, messaging technologies, etc., are contemplated within the scope of the present disclosure. For example, the same approach may be used with ANDROID® devices, iPHONES®, other smartphones, music-playing devices such as an iPOD®, tablet computers, etc.

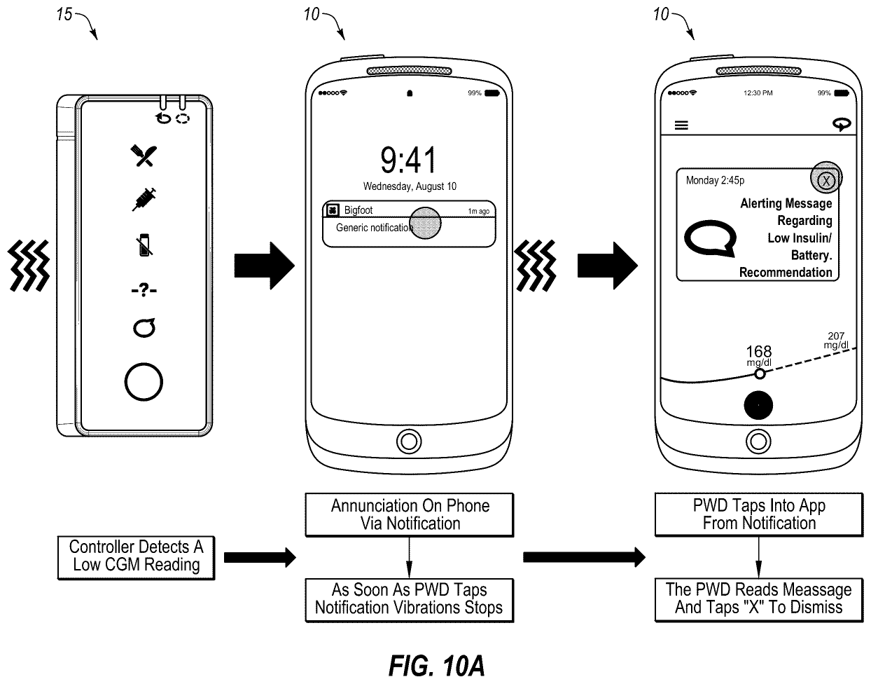

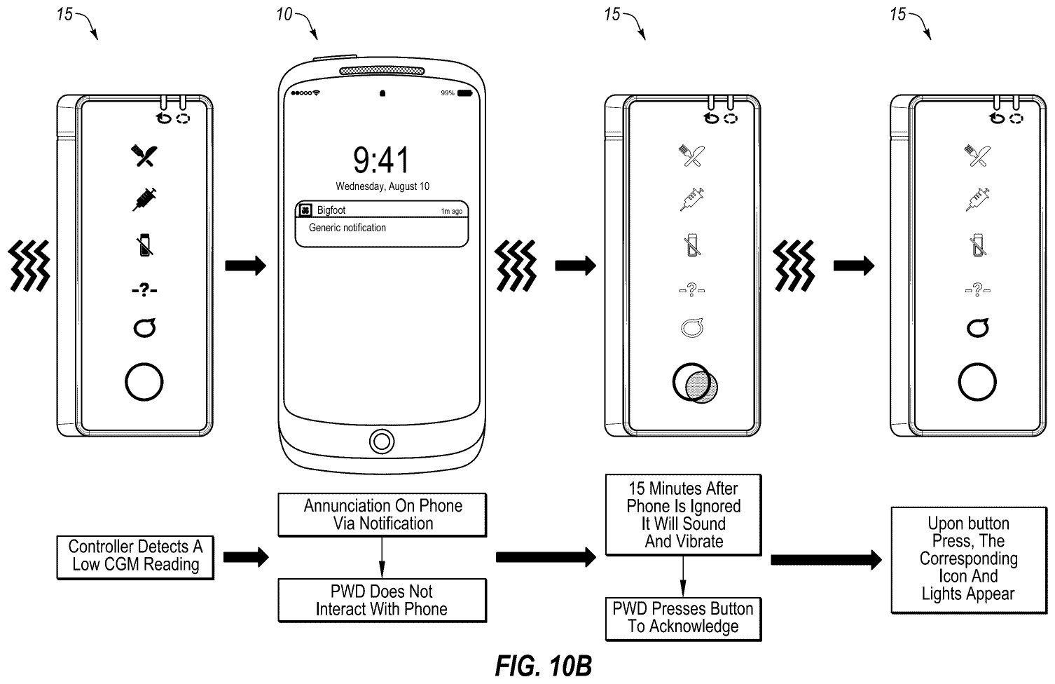

FIG. 10A depicts an example progression of an alert notification regarding battery or insulin levels if the user acknowledges the alert condition within a predetermined period of time. FIG. 10B depicts an example progression of an alert notification regarding battery or insulin levels if the user fails to acknowledge the alarm condition within a predetermined period of time. These progressions are similar to that depicted in FIGS. 9A and 9B for an alarm, but involve an alert message, thus the message icon 245 illuminates, with the automation mode indicator light 236 still illuminated if the alarm is snoozed on the medication delivery device or the status is checked. As noted above, the icon displayed for the alert message on the remote user-interface device matches the icon illuminated on the medication delivery device.

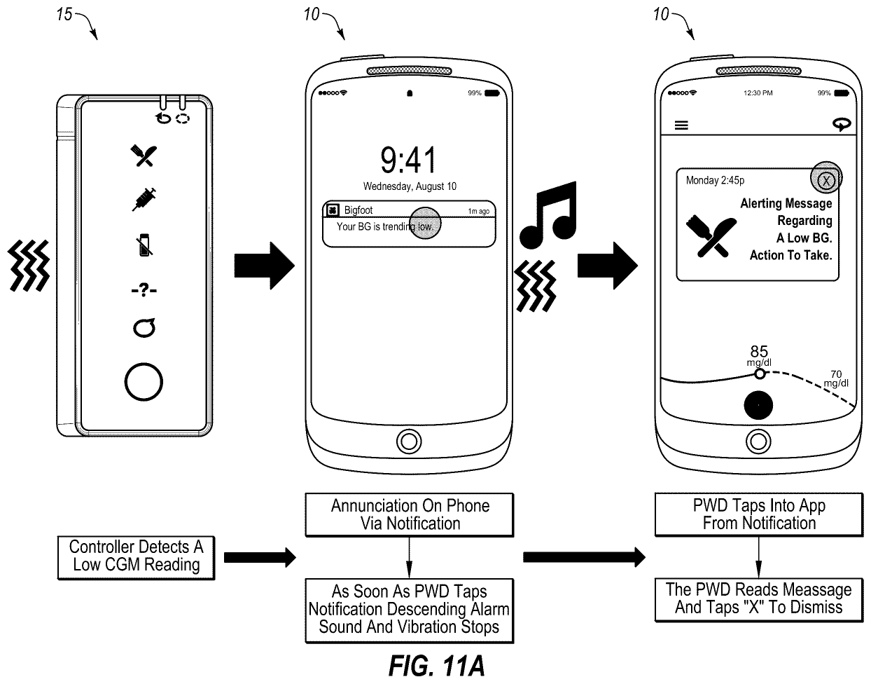

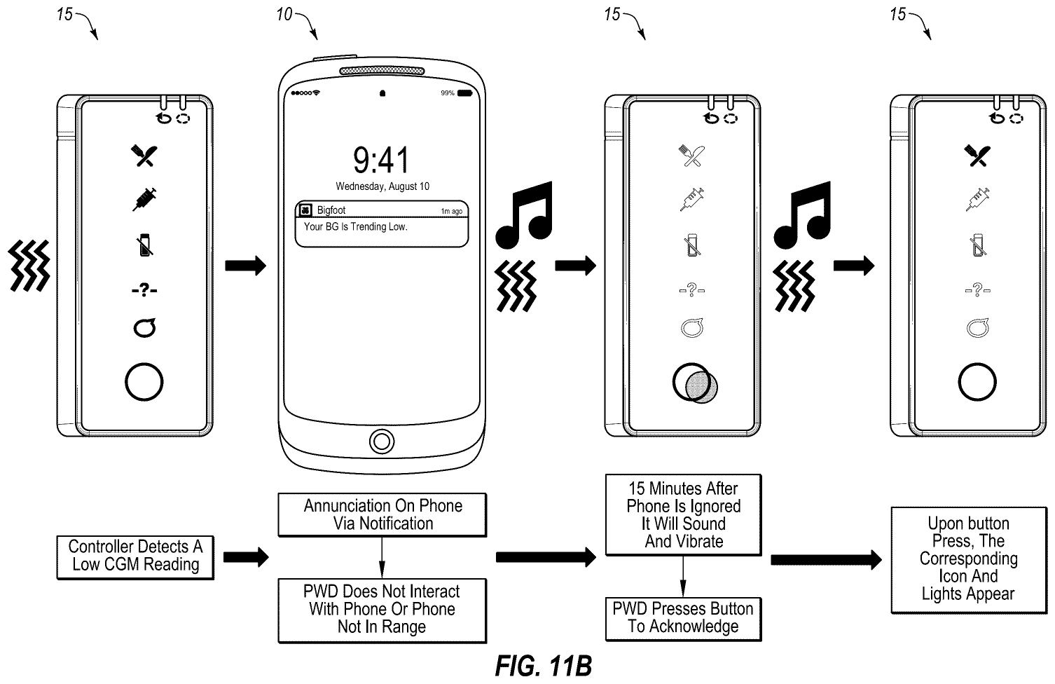

FIG. 11A depicts an example progression of an alert notification regarding a first blood glucose event if the user acknowledges the alert condition within a predetermined period of time. FIG. 11B depicts an example progression of an alert notification regarding a first blood glucose event if the user fails to acknowledge the alarm condition within a predetermined period of time. FIGS. 11A and 11B involve an alert regarding a predicted low glucose event that indicates that the user should consume carbohydrates in order to avoid a hypoglycemic condition. Any suitable predictive technique can be used to trigger this alert, which may escalate to an alarm condition if the user reaches a hypoglycemia threshold. The illuminable icon 241 indicating a need to cat on the medication delivery device is again reinforced on the remote user-interface device by using the same icon in the message.

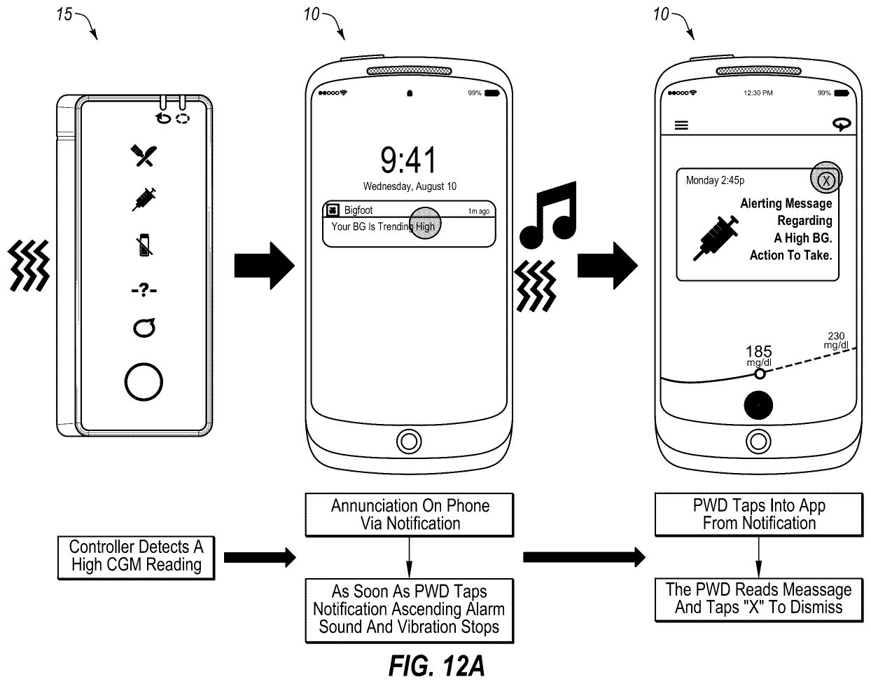

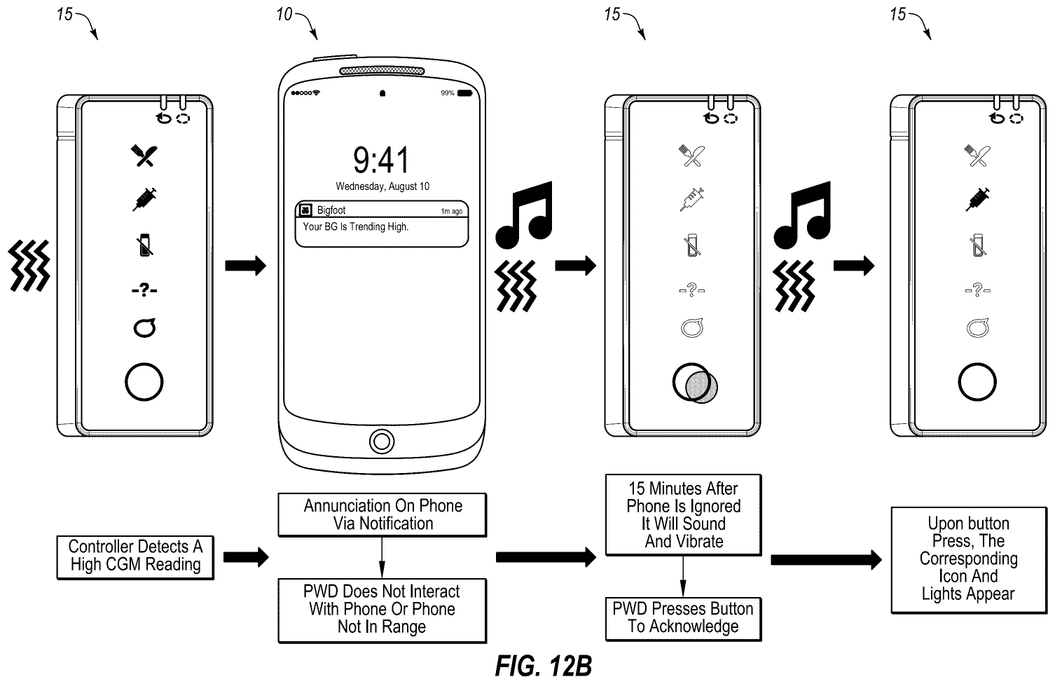

FIG. 12A depicts an example progression of an alert notification regarding a second blood glucose event if the user acknowledges the alert condition within a predetermined period of time. FIG. 12B depicts an example progression of an alert notification regarding a second blood glucose event if the user fails to acknowledge the alarm condition within a predetermined period of time. FIGS. 12A and 12B involve an alert condition where the user is predicted to reach and/or stay in a hypoglycemic state without a corrective dose of insulin. In some cases, this alert condition can consider whether the user has announced and/or bolused for a meal. In some cases, this alert condition may be triggered if a meal is consumed and the user neglected to announce or bolus for the meal. Insulin injection icon 242 can indicate the need for the user to administer a bolus of insulin, and again the use of the same icon on the remote user-interface device 10 can reinforce the types of actions the user should take when seeing the icon when the remote user-interface device is not available.

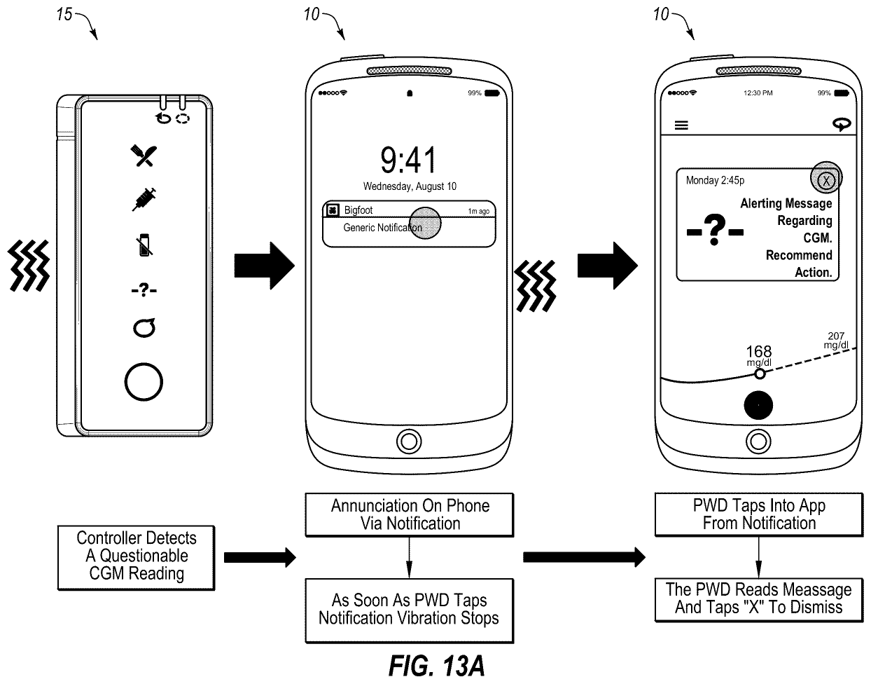

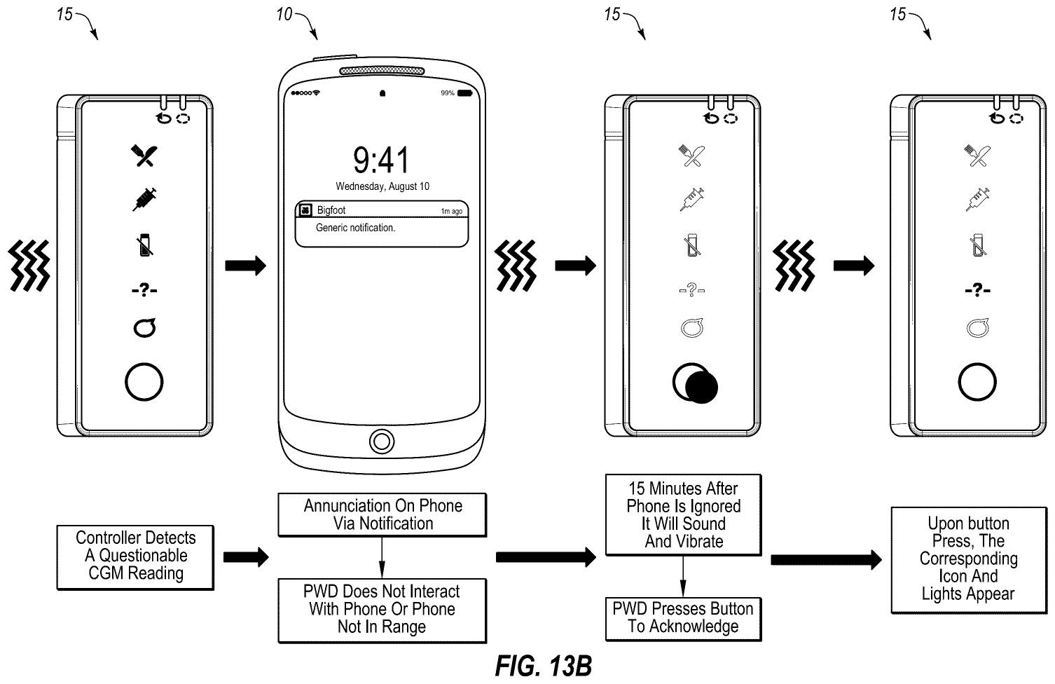

FIG. 13A depicts an example progression of an alert notification regarding a CGM event if the user acknowledges the alert condition within a predetermined period of time.

FIG. 13B depicts an example progression of an alert notification regarding a CGM event if the user fails to acknowledge the alarm condition within a predetermined period of time. The -?-icon 244 can indicate that the CGM is providing questionable or nonexistent data. In some cases, other icons, such as a triple question mark, can be used to indicate that the CGM is providing unreliable data. Again, the meaning of icon 244 is reinforced by the use of the same icon on the remote user-interface device.

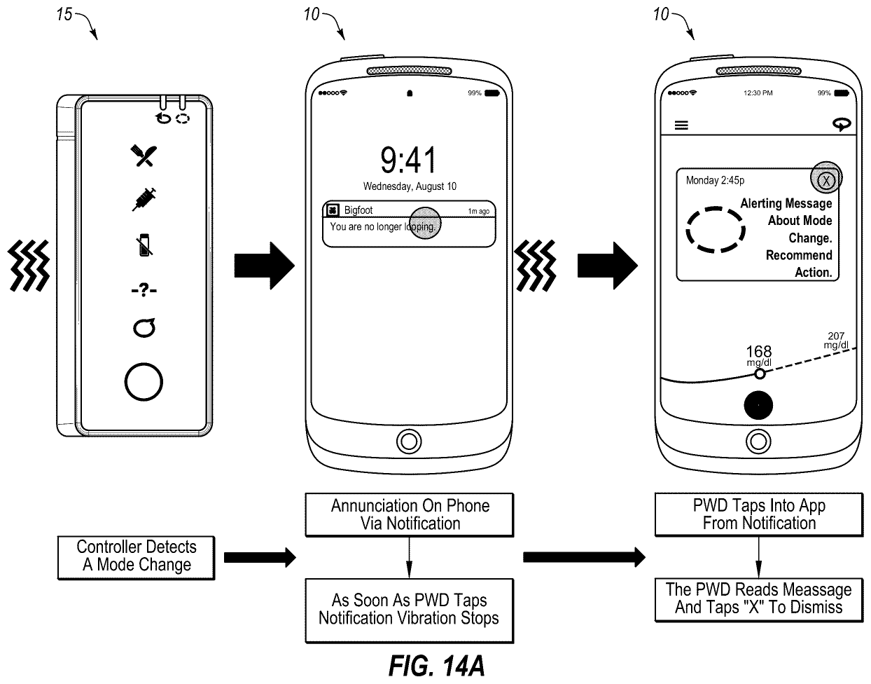

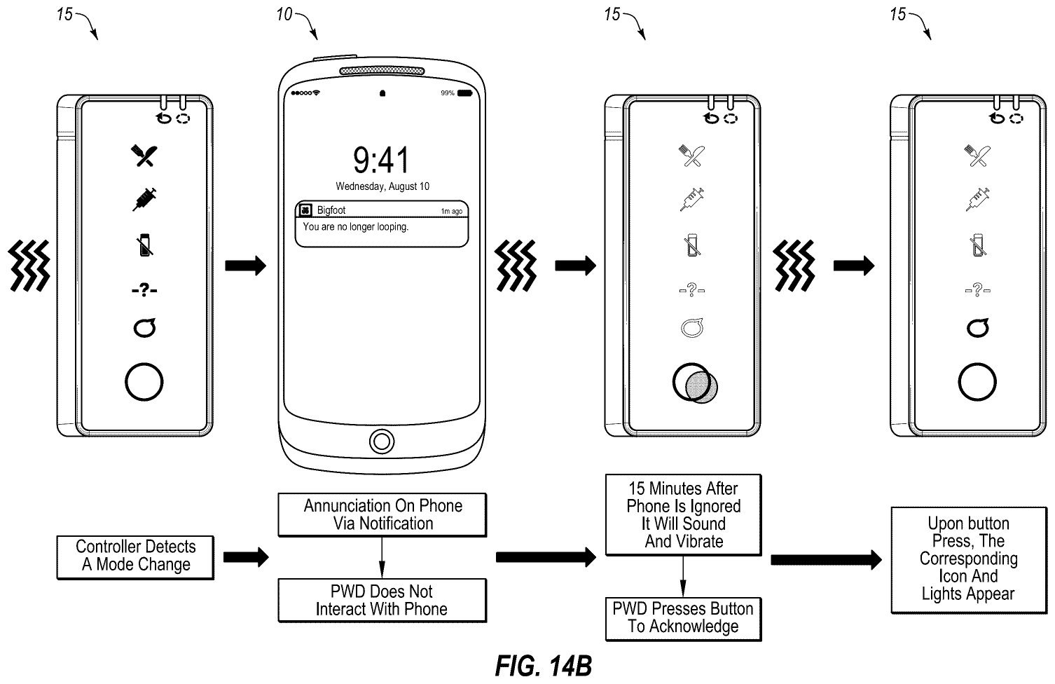

FIG. 14A depicts an example progression of an alert notification regarding a mode change event if the user acknowledges the alert condition within a predetermined period of time. FIG. 14B depicts an example progression of an alert notification regarding a mode change event if the user fails to acknowledge the alarm condition within a predetermined period of time. As shown, message icon 245 is issued along with a change of the mode indicator lights to indicate the current mode.

In some cases, alarms may be accompanied by audible alarms (optionally with haptic feedback) while alerts are accompanied by only haptic feedback. As shown in FIGS. 9-14, medication delivery devices can provide some haptic feedback when they send a wireless communication about an alarm or alert condition to a remote user-interface device, which can help a user understand that an alarm or alert coming from the remote user-interface device is coming from the medication delivery device.

In some cases, mode indicator lights 236 and 237 can be positioned along a top surface of the medication delivery device so that a user having the medication delivery device in their pocket can quickly check to ensure that the system is in automated mode without fully removing the medication delivery device from the user's pocket. It is envisioned that these lights will be the most frequently checked, as it will confirm that the medication delivery device is operational, delivering insulin, and indicate the current mode, while other message, alarms, and alerts can be readily evaluated using a remote user-interface device. Additionally or alternatively, any of the lights or icons may be placed at any location on the surface of the medication delivery device to facilitate observation of the icons or lights. The location and arrangement of the various icons and/or lights may be arranged based on any number of factors, including importance to user safety, frequency of use, etc.

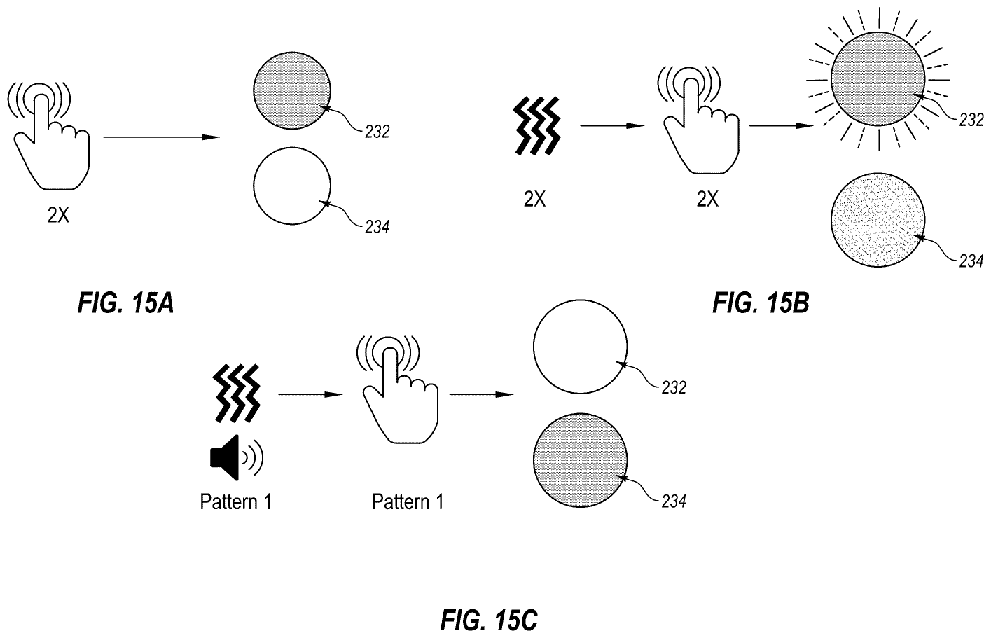

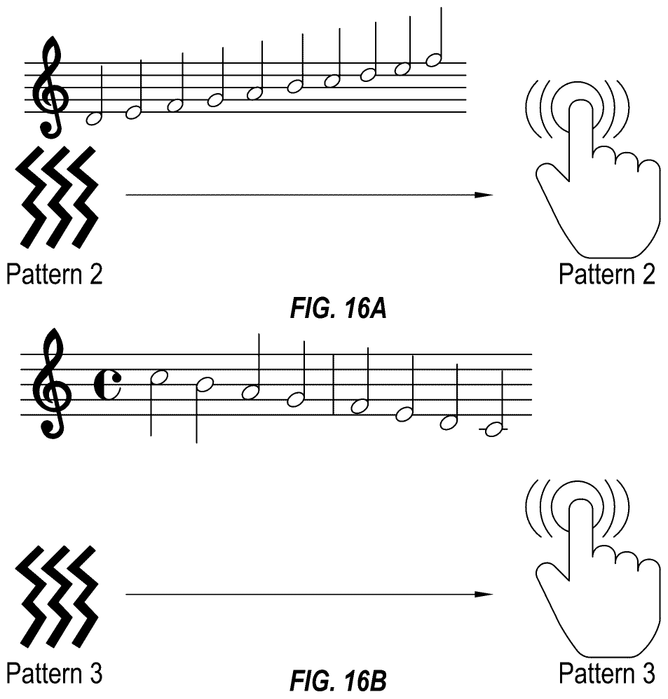

Although FIGS. 2-14 depict a certain configuration and use of notification lights, other light configurations and signaling techniques are also envisioned. For example, FIGS. 15A-15C symbolically illustrate how an example pair of notification lights on an automated medication infusion pump can inform a user about the status of the medication delivery system, even without access to the remote user-interface device 10. By pushing a button on or double tapping on the housing of medication delivery device 15 or 15′ will illuminate medication delivery indicator light 232 if insulin is being delivered. In some cases, medication delivery indicator light 232 can display different colors to indicate a mode of insulin delivery. In some cases, medication delivery indicator light 232 can remain continuously illuminated or blink to indicate a mode of insulin delivery. In some cases, medication delivery indicator light 232 can display different colors or blink using a different frequency to indicate whether real-time analyte sensor data is being received by medication delivery device 15 or 15′. Message indicator light 234 can be included on medication delivery device 15 or 15′ to provide lights indicative of whether there is a message for the user available on remote user-interface device 60. In some cases, the urgency of the message can be conveyed by the color of message indicator light 234 and/or whether message indicator light 234 is blinking. For example, in some cases, message indicator light 234 can be yellow to indicate that a medication delivery device or analyte sensor maintenance activity is due within the next 3 hours. In some cases, message indicator light 234 can illuminate red to indicate that the system requires immediate maintenance and/or that the user has a high or low blood glucose reading. FIG. 15B symbolically illustrates a situation where the analyte sensor and/or the medication delivery device vibrates to indicate the change in mode and/or the presence of a message. As shown, after the vibration pattern, the user can tap the device using a tap pattern (such as a double tap) to stop the vibration and trigger the illumination of indicator lights 232 and 234. In some cases, the vibration will repeat until the user stops the alarm with a tap pattern, which in some cases may be required to match the vibration pattern within a predetermined margin of error. For example, FIGS. 16A and 16B illustrate example alarms and how a user can snooze the alarm or alarms by matching a vibration pattern and/or an alarm pattern (e.g., an alarm pattern of musical notes) with a tap pattern.

As shown in FIG. 15B, medication delivery indicator light 232 is blinking to indicate that the system is not automating medication delivery and message indicator light 234 is yellow to indicate that a message is waiting for the user on the remote user-interface device 10. For example, the message could be a message that the system has entered a non-automated mode because of a loss of analyte sensor data reaching the medication delivery device.

Methods, systems, and devices provided herein can additionally supply an audible alarm or alert to the analyte sensor and/or the medication delivery device (instead of or with vibration) to indicate that the system requires immediate attention. For example, an audible alarm could be triggered if there is an occlusion, if the user has a high or low analyte sensor data point, if the medication delivery device is out of insulin or is expected to be out of insulin in the next hour. FIG. 15C symbolically illustrates a situation where the analyte sensor and/or the medication delivery device vibrates and issues an audible alarm to indicate the need for immediate user interaction with the system. As shown, after the vibration and alarm sound, the user can tap the device using a tap pattern (such as a double tap) to temporarily quiet the vibration and alarm sound and trigger the illumination of indicator lights 232 and 234. In some cases, the tapping will not quiet the alarm if the alarm is more urgent. As shown in FIG. 15C, medication delivery indicator light 232 is off to indicate that the system is not delivering insulin and message indicator light 234 is red to indicate that the system requires immediate attention. In this situation, the user will know that they need to immediately find their remote user-interface device and/or take over responsibility to manage their blood glucose values. In some cases, if the user is unable to find their remote user-interface device, the user will know to check their blood glucose level (e.g., with a blood glucose meter) and find insulin from another source if additional insulin is needed.

Further example embodiments are listed below.

Embodiment 1: An on-body networked medication-delivery system comprising: an analyte sensor adapted to generate analyte data for a user and wirelessly transmit the analyte data; a medication delivery device in wireless communication with the analyte sensor, the medication delivery device comprising: a medication reservoir or a space to receive a medication reservoir; a drive system adapted to meter the administration of medication out of the medication delivery device; a feature to provide audible, visual, or haptic feedback to a user; a controller adapted to change a dosage of medication based at least in part on the analyte sensor data and adapted to issue alarm and alert wireless communications based on a detection of an alarm or alert condition; and a tap detector or button adapted to permit the user to check the status of the medication delivery device or to acknowledge alert or alarm conditions; and a remote user-interface device in wireless communication with the medication delivery device, the remote user-interface device being adapted to receive the alarm and alert wireless communications from the controller and provide an audible, visual, or haptic alarm or alert message to the user and permit the user to acknowledge an associated alarm or alert condition, the remote user-interface device being adapted to wirelessly communicate each acknowledgement to the controller, wherein the controller is adapted to trigger an audible, visual, or haptic alarm or alert message via a feature to provide audible, visual, or haptic feedback if the controller fails to receive an acknowledgement of the alert or alarm condition within a predetermined period of time after the controller issues the alarm and alert wireless communication.

Embodiment 2: The system of Embodiment 1, wherein the system is a diabetes management system, the medication delivery device is an insulin pump, and the analyte sensor is a continuous glucose monitor.

Embodiment 3: The system of Embodiment 1 or Embodiment 2, wherein the medication delivery device is a patch pump.

Embodiment 4: The system of one of Embodiments 1-3, wherein the medication delivery device comprises a durable controller and a disposable pump body, each having a housing and being removably connectable, the disposable pump body comprising at least the medication reservoir or a space to receive a medication reservoir and the durable controller comprising at least the feature(s) to provide audible, visual, or haptic feedback, the controller, and the tap detector or button.

Embodiment 5: The system of one of Embodiments 1-4, wherein the medication delivery device comprises a button.

Embodiment 6: The system of one of Embodiments 1-5, wherein the feature(s) to provide audible, visual, or haptic feedback to a user comprises at least one light associated with an icon.

Embodiment 7: The system of Embodiment 6, wherein the remote user-interface device is adapted to present the icon for an alarm or alert condition.

Embodiment 8: The system of Embodiment 7, wherein the at least one light associated with the icon does not illuminate on the housing until the tap detector detects a tap, or the button is pressed, or until the predetermined period of time.

Embodiment 9: The system of one of Embodiments 1-8, wherein a user can acknowledge an audible, visual, or haptic alarm or alert message provided by a remote user-interface device by tapping the medication delivery device or pressing the button on the medication delivery device even before the medication delivery device triggers the audible, visual, or haptic alarm or alert message via the feature(s) to provide audible, visual, or haptic feedback.

Embodiment 10: The system of one of Embodiments 1-9, wherein the feature(s) to provide audible, visual, or haptic feedback comprises a vibration motor adapted to provide haptic feedback, wherein the controller is adapted to provide haptic feedback or audible feedback, upon issuing the alarm and alert wireless communications, wherein the audible alarm or alert message triggered if the controller fails to receive an acknowledgement of the alert or alarm condition within a predetermined period of time is louder or longer in duration than the haptic feedback or audible feedback provided when the controller issues the alarm and alert wireless communications.

Embodiment 11: The system of one of Embodiments 1-10, wherein the predetermined period of time is at least 30 seconds and no greater than 1 hour, between 1 minute and 30 minutes, between 3 minutes and 20 minutes, or between 5 minutes and 15 minutes, wherein the predetermined period of time for an alarm or alert condition can depend on the alarm or alert condition.

Embodiment 12: The system of one of Embodiments 1-11, wherein an acknowledgement of an alarm or alert will quiet audible or haptic feedback for the alarm or alert condition for a predetermined snooze period of time, wherein the controller is adapted to issue new alarm and alert wireless communications after the predetermined snooze period of time if the alarm or alert condition is still detected as being present.

Embodiment 13: The system of one of Embodiments 1-12, wherein the remote user-interface device is adapted to present the user with troubleshooting instructions using text, audio, or video to remove the alarm or alert condition, wherein the medication delivery device does not present any troubleshooting instructions using text, audio, or video.

Embodiment 14: The system of one of Embodiments 1-13, wherein the medication delivery device comprises a housing that contains a non-rechargeable, non-replaceable battery.

Embodiment 15: The system of one of Embodiments 1-4, wherein the remote user-interface device is adapted to allow a user to send instructions to the medication delivery device using the remote user-interface device, wherein the remote user-interface device can prompt the user to confirm the instructions by pressing the button or tapping the controller under certain conditions.

Embodiment 16: The system of Embodiment 15, wherein the controller is adapted to require a user to confirm a bolus delivery by pressing the button or tapping the controller if the dosage is determined by the controller to be unusual based on typical dosage amounts administered by the user, based on the timing the dosage or the timing of a previous dosage, or based on a prediction of how the dosage will change analyte levels for the user.

Embodiment 17: The system of one of Embodiments 1-16, wherein the medication delivery device comprises one or more icons, and one or more lights associated with those one or more icons, indicating whether the medication is being delivered based on the analyte sensor or not or whether there is an error with the analyte sensor.

Embodiment 18: The system of one of Embodiments 1-17, wherein the medication delivery device comprises one or more icons, and one or more lights associated with those one or more icons, indicating that an amount of medication in the medication delivery device is below a threshold level.

Embodiment 19: The system of one of Embodiments 1-18, wherein the medication delivery device comprises one or more icons, and one or more lights associated with those one or more icons, indicating that the user should administer more medication or consume carbohydrates.

Embodiment 20: The system of one of Embodiments 1-19, wherein the medication delivery device comprises one or more icons, and one or more lights associated with those one or more icons, indicating that a more detailed message for the user is awaiting the user on the remote user-interface device.

Embodiment 21: A method for issuing alarms and alerts in an on-body networked diabetes management system, the method comprising: receiving glucose sensor data from a continuous glucose monitor; determining a dosage of insulin delivery based at least in part on the glucose sensor data; detecting an alarm or alert condition; sending a wireless communication regarding the alarm or alert condition to a remote user-interface device; triggering an audible, visual, or haptic alarm or alert on the insulin delivery device if the insulin delivery device does not receive an acknowledgement of the alarm or alert condition within a predetermined period of time.

Embodiment 22: The method of Embodiment 21, wherein the user can acknowledge the alarm by pressing a button on the insulin delivery device or by tapping the insulin delivery device and by interacting with the remote user-interface device, wherein the insulin delivery device can receive an acknowledgement of the alarm or alert condition as part of a wireless communication from the remote user-interface device.

Embodiment 23: The method of Embodiment 21 or Embodiment 22, further comprising triggering audible or haptic feedback of the insulin delivery device when sending the wireless communication regarding the alarm or alert condition to the remote user-interface device, wherein the audible, visual, or haptic alarm or alert on the insulin delivery device after the predetermined period of time is louder or longer in duration than the feedback initiated when sending the wireless communication.

Embodiment 24: The method of one of Embodiments 21-23, further comprising stopping the audible, visual, or haptic alarm or alert on the insulin delivery device when a button on the insulin delivery device is pressed.

Embodiment 25: The method of Embodiment 24, wherein the button must be pressed at least twice during a predetermined period of time or according to a predetermined pattern for the audible, visual, or haptic alarm or alert to be stopped.

Embodiment 26: The method of Embodiment 24 or 25, wherein stopping the audible, visual, or haptic alarm or alert on the insulin delivery device prevents the triggering of any audible, visual, or haptic alarms or alerts regarding that alarm or alert condition or the sending of any wireless communication regarding the alarm or alert condition for a predetermined period of time, wherein the process of Embodiment 21 will repeat after the predetermined period of time if the alarm or alert condition is present after the predetermined period of time.

Embodiment 27: The method of one of Embodiments 21-26, wherein the alarm or alert condition is an indication of a change from a first mode of operation to a second mode of operation.

Embodiment 28: The method of one of Embodiments 21-26, wherein the alarm or alert condition is an indication of an amount insulin remaining in the insulin delivery device being below a threshold level.

Embodiment 29: The method of one of Embodiments 21-26, wherein the alarm or alert condition is an indication of a low glucose condition or a high glucose condition.

Embodiment 30: The method of Embodiment 29, wherein the audible, visual, haptic alarm or alert on the insulin delivery device includes the illumination of an icon or next to an icon indicating that the user should eat or should administer insulin.

Embodiment 31: The method of one of Embodiments 21-26, wherein the alarm or alert condition is a notice that the continuous glucose monitor is not working, not in range, or not reliable.

Embodiment 32: The method of one of Embodiments 21-26, wherein the alarm or alert condition is a notice about a possible occlusion, a possible air bubble, a possible missed meal announcement, a possible need to change an infusion set, a possible need to calibrate a CGM, a possible need to replace the CGM, or a possible need to check ketone levels, wherein the audible, visual, haptic alarm or alert on the insulin delivery device includes the illumination of an icon or next to an icon indicating that the user should check the remote user-interface device for information about the alert.

Embodiment 33: An insulin delivery device adapted for wireless communication with a continuous glucose monitor and a remote user-interface device, the insulin delivery device comprising: an insulin reservoir or a space to receive an insulin reservoir; a drive system adapted to meter the administration of insulin out of the insulin delivery device; a wireless transmitter and receiver adapted to send and receive wireless communications from at least a continuous glucose monitor and a remote user-interface device; a controller adapted to change a dosage of medication based at least in part on data from the continuous glucose monitor and adapted to issue alarm and alert wireless communications based on a detection of an alarm or alert condition; a housing containing at least the controller and the wireless transmitter and receiver; a tap detector within the housing or a button on the housing adapted to permit the user to check the status of the insulin delivery device or to acknowledge alert or alarm conditions; and one or more lights adapted to illuminate icons or adjacent to icons on the housing indicating a mode of operation of the insulin delivery device and whether insulin is being delivered to the user.

Embodiment 34: The device of Embodiment 33, further comprising one or more lights adapted to illuminate icons or adjacent to icons on the housing indicating that a message is awaiting the user on the remote user-interface device.

Embodiment 35: The device of Embodiment 33 or Embodiment 34, further comprising one or more lights adapted to illuminate icons or adjacent to icons on the housing indicating that the user has a blood glucose condition requiring the consumption of carbohydrates or the administration of additional insulin.

Embodiment 36: The device of Embodiment 35, wherein the user cannot administer additional insulin using the insulin delivery device without accessing the remote user-interface device.

Embodiment 37: The device of Embodiment 36, wherein the controller is adapted to evaluate whether a wireless communication from a remote user-interface device is within one or more predefined parameters.

Embodiment 38: The device of Embodiment 37, wherein the controller is adapted to send a wireless communication to the remote user-interface device indicating that a bolus is outside of one or more predefined parameters, or indicating the user must confirm the bolus on the insulin delivery device by tapping or pressing the button.

Embodiment 39: The device of one of Embodiments 33-38, further comprising one or more lights adapted to illuminate icons or adjacent to icons on the housing indicating that the insulin delivery device has less than a threshold amount of insulin remaining.

Embodiment 40: The device of one of Embodiments 33-39, further comprising one or more lights adapted to illuminate icons or adjacent to icons on the housing indicating that there is a problem with the data being received, or a lack of data being received, from the continuous glucose monitor.

Embodiment 41: A medication delivery system comprising a medication delivery device and a remote user-interface device, the medication delivery device and the remote user-interface device being in wireless communication, the medication delivery device being adapted to automatically administer medication according to programmed rate, a programmed schedule, or based on analyte sensor data without user input, the remote user-interface device being adapted to receive user commands for the medication delivery device to administer additional doses of medication, adjust the programmed delivery rate or schedule, or adjust an algorithm that determines a dosage based on the analyte sensor data, wherein both the remote user-interface device and the medication delivery device are adapted to provide audible, visual, or haptic feedback to issue an alarm or alert regarding the ability of the medication delivery device to deliver medication, wherein the medication delivery device is adapted to detect a condition that prevents the delivery of medication and is adapted to send an alarm wireless communication to the remote user-interface device regarding the condition, wherein the remote user-interface device issues an audible, visual, or haptic alarm when the alarm wireless communication is received and provides a feature for the user to acknowledge the alarm, wherein the remote user-interface device sends an acknowledgement wireless communication to the medication delivery device upon the user acknowledging the alarm, wherein the medication delivery device is adapted to issue an audible, visual, or haptic alarm after a predetermined period of time after the alarm wireless communication is sent unless the medication delivery device receives the acknowledgement wireless communication during the predetermined period of time.

Embodiment 42: The medication delivery system of Embodiment 41, wherein the medication delivery device includes a feature to receive a user's acknowledgement of an audible, visual, or haptic alarm to silence the alarm.

Embodiment 43: The medication delivery system of Embodiment 41 or Embodiment 42, wherein the medication delivery device is an insulin infusion pump, wherein the medication is insulin, and wherein the remote user-interface device is a smartphone.

Embodiment 44: The medication delivery system of Embodiment 43, further comprising a continuous glucose monitor in wireless communication with the insulin infusion pump, wherein the insulin infusion pump delivers different amounts or rates of insulin based on glucose data from the continuous glucose monitor.

Embodiment 45: The medication delivery system of Embodiment 44, wherein the insulin infusion pump is not adapted to display specific concentrations of the glucose data, but is adapted to send glucose data wireless communications to the smartphone, wherein the smartphone is adapted to display specific concentrations of the glucose data.

Embodiment 46: The medication delivery system of Embodiment 45, wherein the insulin infusion pump is adapted to illuminate one or more icons, or a light next to one or more icons, that indicate that the user is in or expected to experience hypoglycemic state or a hyperglycemic state, that indicate that the user should administer more insulin, or that indicate that the user should consume food, wherein the light becomes illuminated when the insulin infusion pump issues an issue an audible, visual, or haptic alarm or when the user acknowledges the alarm on the insulin infusion pump.

Embodiment 47: The medication delivery system of one of Embodiments 43-46, wherein the insulin infusion pump is adapted to illuminate one or more icons, or a light next to one or more icons, that indicate that the user is in out of insulin, wherein the light becomes illuminated when the insulin infusion pump issues an issue an audible, visual, or haptic alarm or when the user acknowledges the alarm on the insulin infusion pump.

While certain embodiments have been described and shown in the accompanying drawings, such embodiments are merely illustrative and not restrictive of the scope of the disclosure, and this disclosure is not limited to the specific constructions and arrangements shown and described, since various other additions and modifications to, and deletions from, the described embodiments will be apparent to one of ordinary skill in the art. Thus, the scope of the disclosure is only limited by the literal language, and legal equivalents, of the claims that follow.

What is claimed is:

1. A networked medication-delivery system comprising:
an analyte sensor adapted to generate analyte data about a patient and transmit the analyte data;
a medication delivery device in communication with the analyte sensor; and
a remote user-interface device in communication with the medication delivery device,
wherein the remote user-interface device is configured to:
receive data regarding a status condition from the medication delivery device; and
present, in a persistent manner, an indication of the status condition within a visual user interface (UI) element until closed in response to a predefined action.

2. The networked medication-delivery system of claim 1, wherein the remote user-interface device is configured to clear the indication of the status condition at least partially responsive to indication of occurrence of the predefined action.

3. The networked medication-delivery system of claim 1, wherein the remote user-interface device is configured to stack multiple visual UI elements, including the visual UI element, the stacked multiple visual UI elements respectively including different indications of status conditions.

4. The networked medication-delivery system of claim 3, wherein the remote user-interface device is configured to stack the multiple visual UI elements in an arrangement that ensures respective ones of the stacked multiple visual UI elements are at least partially visible.

5. The networked medication-delivery system of claim 1, wherein the remote user-interface device is configured to continue to present, in the persistent manner, the indication of the status condition within the visual UI element after user acknowledgment of the status condition.

6. The networked medication-delivery system of claim 1, wherein the remote user-interface device is configured to continue to present, in the persistent manner, the indication of the status condition within the visual UI element despite user interaction with remote-interface device.

7. The networked medication-delivery system of claim 6, wherein the user interaction includes one or more of: adjusting therapy settings or acknowledging an alarm.

8. The networked medication-delivery system of claim 1, wherein the indication of the status condition within the visual UI element is continuously visible on a screen while presented in the persistent manner.

9. The networked medication-delivery system of claim 1, wherein the remote user-interface device is configured to present a snooze or mute alarm option, and wherein the remote user-interface device is configured to continue to present, in the persistent manner, the indication of the status condition within the visual UI element upon activation of the snooze or moot alarm option.

10. A method, comprising:
receiving data regarding a status condition from a medication delivery device at a remote user-interface device; and
presenting, in a persistent manner, an indication of the status condition within a visual user interface (UI) element on the remote user-interface device until the condition is closed in response to a predefined action.

11. The method of claim 10, comprising:
clearing the indication of the status condition on the remote user-interface device at least partially in response to an occurrence of the predefined action.

12. The method of claim 10, comprising:
stacking multiple visual UI elements, including the visual UI element, on the remote user-interface device, where the stacked multiple visual UI elements respectively include different indications of status conditions.

13. The method of claim 10, comprising:
stacking multiple visual UI elements in an arrangement on the remote user-interface device that ensures respective ones of the stacked multiple visual UI elements are at least partially visible.

14. The method of claim 10, comprising:
continuing presenting, in a persistent manner, the indication of the status condition within the visual UI element on the remote user-interface device after user acknowledgment of the status condition.

15. The method of claim 10, comprising:
continuing presenting, in a persistent manner, the indication of the status condition within the visual UI element on the remote user-interface device while other user interface elements are changing in response to user interaction with the remote user-interface device.

16. The method of claim 15, wherein the user interaction in response to which other user interface elements are changing is a change to therapy settings.

17. The method of claim 10, comprising:
ensuring that the indication of the status condition within the visual UI element is continuously visible on a screen while being presented in the persistent manner.

18. The method of claim 10, comprising:
presenting a snooze or mute alarm option; and
continuing presenting, in the persistent manner, the indication of the status condition within the visual UI element upon activation of the snooze or moot alarm option.
